(19) United States
(12) Patent Application Publication
(10) Pub. No.: US 2024/0207311 A1
(43) Pub. Date: Jun. 27, 2024

(54) TUMOR INFILTRATING LYMPHOCYTES THERAPY

(71) Applicant: Alethia Biotherapeutics Inc., Montréal (CA)

(72) Inventor: Mario FILION, Longueuil (CA)

(73) Assignee: Alethia Biotherapeutics Inc., Montréal, QC (CA)

(21) Appl. No.: 18/287,334

(22) PCT Filed: Apr. 27, 2022
(86) PCT No.: PCT/CA2022/050636

§ 371 Date: Oct. 18, 2023

Related U.S. Application Data
(60) Provisional application No. 63/180,279, filed on Apr. 27, 2021.

(51) Int. Cl. A61K 35/17; A61K 31/337; A61K 39/00; A61P 35/04; C07K 16/30; C12N 5/0783

(52) U.S. Cl. CPC A61K 35/17; A61K 31/337; A61K 39/4611; A61K 39/4631; A61K 39/464402; A61P 35/04; C07K 16/30; C12N 5/0636; A61K 2039/505; A61K 2239/13; A61K 2239/38

(57) ABSTRACT

The present disclosure generally relates to a method of treating cancer by administration of autologous tumor infiltrating lymphocytes (TILs) isolated from a subject that has received prior treatment with an anti-cancer therapy that comprises an anti-clusterin antibody or antigen binding fragment thereof. The method of the present disclosure comprises administering the anti-cancer therapy to the subject, isolating TILs. and reinfusing TILs to the subject. The present disclosure also relates to the use of an anti-clusterin antibody or antigen binding fragment thereof in an in vitro or ex vivo method of generating tumor infiltrating lymphocytes (TILs).

### TECHNICAL FIELD

The present disclosure generally relates to a method of treating cancer by administration of autologous tumor infiltrating lymphocytes (TILs) isolated from a subject that has received prior treatment with an anti-cancer therapy that comprises an anti-clusterin antibody or antigen binding fragment thereof. The method of the present disclosure comprises administering the anti-cancer therapy to the subject, isolating TILs and reinfusing TILs to the subject. The present disclosure also relates to the use of an anti-clusterin antibody or antigen binding fragment thereof in an in vitro or ex vivo method of generating tumor infiltrating lymphocytes (TILs).

### BACKGROUND

Immune cell therapies of solid tumors consist of two different approaches: adoptive transfer of naturally-occurring tumor-specific T cells isolated from tumor infiltrates (TILs) or transfer of genetically-modified T lymphocytes that express a transgenic T-cell receptor (tg-TCR) specific for a tumor antigen, or a chimeric antigen receptor (CAR) composed of a single-chain variable regions of a monoclonal antibody fused to endo-domains of T-cell signaling molecules.

TILs therapy has a long history of development with multiple clinical trials in centers around the world that consistently have demonstrated long-lasting clinical response rates (˜50%) in advanced melanoma and, more recently, in cervical cancer.

A clear advantage of TIL treatment is the broad nature of the T-cell recognition against both defined and un-defined tumors antigens, and in the context of all possible MHC molecules, rather than the single specificity of tg-TCR- or CAR-transduced T cells, and the limited MHC coverage of tg-TCR T cells. On-target/off-tumor toxicity is relatively infrequent in TIL therapy while it is a major problem encountered with genetically modified T-cell therapies.

Treatment of refractory cancers using adoptive transfer of TILs thus represents a powerful approach to therapy for patients with poor prognoses. Gattinoni, et al., Nat. Rev. Immunol. 2006, 6, 383-393. IL-2-based TIL expansion followed by a “rapid expansion process” (REP) has become a preferred method for TIL expansion because of its speed and efficiency (Dudley, et al., Science 2002, 298, 850-54: Dudley, et al., J. Clin. Oncol. 2005, 23, 2346-57: Dudley, et al., J. Clin. Oncol. 2008, 26, 5233-39; Riddell, et al., Science 1992, 257, 238-41: Dudley, et al., J. Immunother. 2003, 26, 332-42). REP can result in a 1,000-fold expansion of TILs over a 14-day period, although it requires a large excess (e.g., 200-fold) of irradiated allogeneic peripheral blood mononuclear cells is (PBMCs, also known as mononuclear cells (MNCs)), often from multiple donors, as feeder cells, as well as anti-CD3 antibody (OKT3) and high doses of interleukin 2 (IL-2) (Dudley, et al., J. Immunother. 2003, 26, 332-42). TILs that have undergone a REP procedure have produced successful adoptive cell therapy following host immunosuppression in patients with melanoma. Current infusion acceptance parameters rely on readouts of the composition of TILs (e.g., CD28, CD8, or CD4 positivity) and on fold expansion and viability of the REP product.

Methods for preparing and expanding TILs are described for example and without limitations in Jin J., et al., J Immunother. 35(3):283-292, 2012, in Dudley, M. E. et al., J Immunother. 26(4): 332-342, 2003, in international application No. PCT/US2018/01633 filed on Jan. 5, 2018 and published on Oct. 4, 2018 under No. WO2018/182817 and in international application No. PCT/US2019/052681 filed on Sep. 24, 2019 and published on Apr. 2, 2020 under No. WO2020/068816, international patent application No. PCT/US2015/025313 filed on Apr. 10, 2015 and published on Oct. 15, 2015 under No. WO2015/157636, international application No. PCT/US2019/052681 filed on Sep. 24, 2019 and published on Apr. 2, 2020 under No. WO2020/068816 (the entire content of all of which is incorporated herein by reference).

Unfortunately, some patients carry tumors that are poorly infiltrated by immune cells and adoptive cell therapy is therefore expected to be of limited utility.

There remains a need to increase the presence of TILs in tumors for modulation of an in vivo antitumor immune response and for adoptive cell therapy.

### SUMMARY

The Applicant came to the unexpected discovery that treatment with an anti-clusterin antibody or antigen binding fragment thereof leads to increased intra-tumor immune infiltration (see international patent application No. PCT/CA2021/050572 filed on Apr. 27, 2021, and international patent application No. PCT/CA2022/050632 filed on Apr. 26, 2022, the entire content of each of which is incorporated herein by reference).

Preliminary data of a phase II clinical trial aimed at evaluating a combination treatment comprising an anti-clusterin antibody (AB-16B5, a.k.a., humanized 16B5) and docetaxel in subjects with metastatic non-small cell lung cancer (NCT04364620), show similar intra-tumor immune cells infiltration (see international patent application No. PCT/CA2022/050632 filed on Apr. 26, 2022).

An anti-cancer therapy comprising an anti-clusterin antibody or antigen binding fragment thereof may thus be administered to a subject having cancer to promote infiltration of immune cells in the tumor microenvironment. Preparations of tumor infiltrating lymphocytes are then generated from tumors of treated subjects for use in adoptive cell therapy.

The present disclosure provides a method of treating a subject having cancer, which comprises a step of administering an anti-cancer therapy comprising an anti-clusterin antibody or antigen binding fragment thereof to the subject, a step of isolating and expanding tumor infiltrating lymphocytes (TILs) from the subject's tumor and a step of reinfusing a preparation of TILs to the subject.

In some embodiments, the method involves administering the preparation of TILs disclosed herein. In some embodiments, the preparation of TILs is composed of one or more TILs culture. In some embodiments, the preparation of TILs is a TILs culture.

In accordance with the present disclosure, the anti-cancer therapy consists of an anti-clusterin antibody or antigen binding fragment thereof provided as a single anti-cancer agent.

In accordance with the present disclosure, the anti-cancer therapy comprises an anti-clusterin antibody or antigen binding fragment thereof and another anti-cancer agent. Accordingly, the anti-cancer therapy may be a combination therapy.

In some embodiments, the combination therapy comprises the anti-clusterin antibody or antigen binding fragment thereof and radiation therapy.

In other embodiments, the combination therapy comprises the anti-clusterin antibody or antigen binding fragment thereof and chemotherapy.

The present disclosure also provides a method of treating cancer with tumor infiltrating lymphocytes (TILs) isolated and expanded from a tumor isolated from a subject treated with an anti-cancer therapy that comprises an anti-clusterin antibody or antigen binding fragment thereof.

In some embodiments, the subject receives lymphocyte-depleting preparative regimen prior to infusion of TILs.

In some embodiments, TILs are isolated and expanded by an in vitro or ex vivo method of generating tumor infiltrating lymphocytes so as to generate a preparation of TILs. In some embodiment, the method involves culturing TILs.

In some embodiments, the method may include a step of removing tumor cells from the TILs culture.

In some embodiments, the method may include a step of selecting CD45⁺ cells from the TILs culture.

In some embodiments, the method may include a step of selecting CD3⁺ cells from the TILs culture.

In some embodiments, the method may include a step of selecting CD4⁺ cells from the TILs culture.

In some embodiments, the method may include a step of selecting CD8⁺ cells from the TILs culture.

In some embodiments, the method may include a step of selecting cells that have an intermediate to high level of INFγ secretion.

In some embodiment, the TILs are selected for their anti-tumor activity in vitro.

Typically, TILs having anti-tumor activity are selected for use in autologous adoptive cell therapy.

In some embodiments, TILs are isolated from a subject that has been treated or is treated with an anti-cancer therapy that comprises an anti-clusterin antibody or an antigen binding fragment thereof as a single agent.

In some embodiments, TILs are isolated from a subject that has been treated or is treated with a combination therapy comprising an anti-clusterin antibody or an antigen binding fragment thereof and a chemotherapeutic agent.

Exemplary embodiments of chemotherapeutic agents include an alkylating agent, an anti-metabolite, an alkaloid, an anti-tumor antibiotic or combination thereof.

In some instances, the alkylating agent may be selected, for example, from altretamine, busulfan, carboplatin, carmustine, cisplatin, cyclophosphamide, dacarbazine, ifosfamide, lomustine, melphalan, temozolomide, trabectedin or derivatives or analogs thereof.

In some instances, the anti-metabolite may be selected, for example, 5-fluorouracil, 6-mercaptopurine, azacytidine, capecitabine, clofarabine, cytarabine, floxuridine, fludarabine, gemcitabine, methotrexate, pemetrexed, pentostatin, pralatrexate, trifluridine, tipiracil or derivatives or analogs thereof.

In some instances, the alkaloid may be selected, for example, from vincristine, vinblastine, vinorelbine, taxanes, etoposide, teniposide, irinotecan, topotecan or derivatives or analogs thereof.

Exemplary embodiments of taxane includes docetaxel, paclitaxel and derivatives or analogues including for example and without limitations, Abraxane®, Cabazitaxel, larotaxel, milataxel, ortataxel, tesetaxel and others described in Ojima et al., Expert Opin Ther Pat. 2016: 26(1): 1-20, the entire content of which is incorporated herein by reference.

In some instances, the anti-tumor antibiotic may be selected, for example, from daunorubicin, doxorubicin, doxorubicin liposomal, epirubicin, idarubicin, valrubicin, derivatives or analogs thereof.

In some embodiments, the chemotherapeutic agent is docetaxel.

In some embodiments, the chemotherapeutic agent is paclitaxel.

In some embodiments, the tumor is resectable.

In some embodiments, the subject has a functional immune system.

In some embodiments, the TILs are obtained from a tumor or tumor fragments isolated by biopsy.

In some embodiment, the TILs are obtained by a method that comprises an initial culture phase and an expansion phase.

In accordance with the present disclosure, the in vitro or ex vivo method of generating tumor infiltrating lymphocytes may comprise a step of contacting tumor fragments with an anti-clusterin antibody or antigen binding fragment thereof.

In accordance with the present disclosure, the anti-clusterin antibody or an antigen binding fragment thereof may be present and/or maintained during the initial culture phase of the method of generating tumor infiltrating lymphocytes.

Alternatively, in accordance with the present disclosure, the anti-clusterin antibody or an antigen binding fragment thereof may be present and/or maintained during the expansion phase of the method of generating tumor infiltrating lymphocytes.

In some embodiment, the method of the present disclosure may involve administering TILs that are not genetically modified. However, it is possible to genetically modify TILs to make them express or overexpress proteins or peptides.

In some embodiments, the preparation of TILs is not genetically modified.

In some embodiments, the preparation of TILs comprises TILs that are genetically modified.

In some embodiments, the preparation of TILs comprises TILs that express a chimeric antigen receptor.

In some embodiments, the preparation of TILs comprises TILs that express a transgenic T-cell receptor.

In some embodiments, the preparation of TILs comprises TILs that are isolated from a primary tumor.

In some embodiments, the preparation of TILs comprises TILs that are isolated from a metastasis.

In accordance with the present disclosure TILs may be isolated from a subject that has received a prior treatment with an anti-cancer therapy as described herein.

In an exemplary embodiment, the subject may have received prior treatment with an anti-clusterin antibody or antigen binding fragment thereof and a taxane such as for example, docetaxel or paclitaxel.

In accordance with the present disclosure, the anti-clusterin antibody or antigen binding fragment thereof may be administered at a dose and/or an administration interval and/or for a treatment period sufficient to result in infiltration of immune cells in the tumor microenvironment.

In accordance with the present disclosure, docetaxel may be administered at a dose and/or an administration interval and/or for a treatment period sufficient to allow chemotherapy-induced immunogenic modulation of tumor.

In accordance with the present disclosure, the method comprises administering an anti-clusterin antibody or antigen binding fragment thereof comprising a light chain variable region comprising the complementarity determining regions (CDRs) of the light chain variable region set forth in SEQ ID NO:9 and a heavy chain variable region comprising the CDRs of the heavy chain variable region set forth in SEQ ID NO: 10.

In accordance with the present disclosure, the method comprises administering an anti-clusterin antibody or antigen binding fragment thereof comprising a light chain variable region having an amino acid sequence having at least 80% identity with the amino acid sequence set forth in SEQ ID NO:9 and a heavy chain variable region having an amino acid sequence at least 80% identity with the amino acid sequence set forth in SEQ ID NO: 10.

In accordance with the present disclosure, the method comprises administering an anti-clusterin antibody or antigen binding fragment thereof comprising a light chain having an amino acid sequence having at least 80% identity with the amino acid sequence set forth in SEQ ID NO:11 and a heavy chain having an amino acid sequence having at least 80% identity with the amino acid sequence set forth in SEQ ID NO: 12.

In accordance with the present disclosure, the method comprises administering an anti-clusterin antibody or antigen binding fragment thereof comprising a light chain variable region having an amino acid sequence set forth in SEQ ID NO:9 and a heavy chain variable region having an amino acid sequence set forth in SEQ ID NO: 10 for the binding of clusterin.

In some embodiments, the anti-clusterin antibody or antigen binding fragment thereof is administered prior to isolating the TILs. In some embodiments, the anti-clusterin antibody or antigen binding fragment thereof and chemotherapeutic agent are administered prior to isolating the TILs. In some embodiments, one or more treatment cycles are administered prior to isolating the TILs.

In some embodiments, the anti-cancer therapy described herein may also be administered after adoptive cell therapy.

In some embodiments, the anti-clusterin antibody or antigen binding fragment thereof is administered after the preparation of TILs is infused. In some embodiments, the anti-clusterin antibody or antigen binding fragment thereof and chemotherapeutic agent are administered after TILs are infused. In some embodiments, one or more treatment cycles are administered after TILs are infused.

In some embodiments, the anti-clusterin antibody or antigen binding fragment thereof is administered at a dose of between approximately 3 mg/kg to approximately 20 mg/kg prior to isolation of TILs or after infusion of TILs.

In some embodiments, the anti-clusterin antibody or antigen binding fragment thereof is administered at a dose of approximately 6 mg/kg.

In some embodiments, the anti-clusterin antibody or antigen binding fragment thereof is administered at a dose of approximately 9 mg/kg.

In some embodiments, the anti-clusterin antibody or antigen binding fragment thereof is administered at a dose of approximately 12 mg/kg.

In some embodiments, the anti-clusterin antibody or antigen binding fragment thereof is administered weekly.

In some embodiments, docetaxel is administered at a dose of between approximately 60 mg/m² to approximately 100 mg/m² prior to isolation of TILs or after infusion of TILs. In some embodiments, docetaxel is administered once every three weeks.

In some embodiments, docetaxel is administered at a dose of approximately 60 mg/m². In some embodiments, docetaxel is administered at a dose of approximately 75 mg/m².

In some embodiments, the subject is treated with the anti-clusterin antibody or antigen binding fragment thereof at a dose of approximately 12 mg/kg once weekly and docetaxel at a dose of approximately 75 mg/m² once every three weeks.

In some embodiments, the subject is treated with the anti-clusterin antibody or antigen binding fragment thereof at a dose of approximately 12 mg/kg once weekly and docetaxel at a dose of approximately 60 mg/m² once every three weeks.

In some embodiments, the subject is treated with the anti-clusterin antibody or antigen binding fragment thereof at a dose of approximately 9 mg/kg once weekly and docetaxel at a dose of approximately 75 mg/m² once every three weeks.

In some embodiments, the subject is treated with the anti-clusterin antibody or antigen binding fragment thereof at a dose of approximately 9 mg/kg once weekly and docetaxel at a dose of approximately 60 mg/m² once every three weeks.

In some embodiments, the subject is treated with the anti-clusterin antibody or antigen binding fragment thereof at a dose of approximately 6 mg/kg once weekly and docetaxel at a dose of approximately 75 mg/m² once every three weeks.

In some embodiments, the subject is treated with the anti-clusterin antibody or antigen binding fragment thereof at a dose of approximately 6 mg/kg once weekly and docetaxel at a dose of approximately 60 mg/m² once every three weeks.

In some embodiments, the subject is treated with the anti-clusterin antibody or antigen binding fragment thereof at a dose of approximately 3 mg/kg once weekly and docetaxel at a dose of approximately 75 mg/m² once every three weeks.

In some embodiments, the subject is treated with the anti-clusterin antibody or antigen binding fragment thereof at a dose of approximately 3 mg/kg once weekly and docetaxel at a dose of approximately 60 mg/m² once every three weeks.

In some embodiments, the anti-clusterin antibody or antigen binding fragment thereof and docetaxel are administered on same day.

In some embodiments, the anti-clusterin antibody or antigen binding fragment thereof and/or docetaxel is administered by infusion over approximately a 1-hour time frame.

In some embodiments, the method of the present disclosure is for treatment of a subject as described herein.

In some embodiments, the subject has a carcinoma.

In some embodiments, the subject has metastatic carcinoma.

In some embodiments, the subject has an endometrial cancer, a breast cancer, a liver cancer, a prostate cancer, a renal cancer, a bladder cancer, a cervical cancer, an ovarian cancer, a colorectal cancer, a pancreatic cancer, a lung cancer, a gastric cancer, a head and neck cancer, a thyroid cancer, a cholangiocarcinoma, a mesothelioma or a melanoma.

In some embodiments, the subject has a metastatic endometrial cancer, a metastatic breast cancer, a metastatic liver cancer, a metastatic prostate cancer, a metastatic renal cancer, a metastatic bladder cancer, a metastatic cervical cancer, a metastatic ovarian cancer, a metastatic colorectal cancer, a metastatic pancreatic cancer, a metastatic lung cancer, a metastatic gastric cancer, a metastatic head and neck cancer, a metastatic thyroid cancer, a metastatic cholangiocarcinoma, a metastatic mesothelioma or a metastatic melanoma.

In some embodiments, the subject is not immunosuppressed or has not received an immunosuppressive medication within 14, 13, 12, 11, 10, 9, 8, 7, 6, 5, 4, 3, 2 days or 1 day prior to treatment with the anti-clusterin antibody or antigen binding fragment thereof or prior to treatment with the anti-clusterin antibody or antigen binding fragment thereof and docetaxel combination therapy.

In some embodiments, the subject is a human subject.

The method of the present disclosure may result in a preparation of TILs or TILs culture that comprises CD4⁺ T cells.

The method of the present disclosure may result in a preparation of TILs or TILs culture that comprises CD8⁺ T cells.

The method of the present disclosure may result in a preparation of TILs or TILs culture that comprises B cells.

The method of the present disclosure may result in a preparation of TILs or TILs culture that comprises NK cells.

The method of the present disclosure may result in a preparation of TILs or TILs culture that comprises NK T cells

The method of the present disclosure may result in a preparation of TILs or TILs culture that has anti-tumor activity.

The present disclosure therefore provides a preparation of tumor infiltrating lymphocytes (TILs) obtained by the method described herein.

The present disclosure therefore provides a tumor infiltrating lymphocytes (TILs) culture obtained by the method described herein.

Accordingly, the present disclosure also provides a preparation of tumor infiltrating lymphocytes (TILs) obtained by a method of treating a subject having cancer with an anti-cancer therapy comprising an anti-clusterin antibody or antigen binding fragment thereof.

In some embodiments, the preparation of TILs is a preparation of expanded TILs.

The present disclosure also provides a TILs culture obtained by a method of treating a subject having cancer with an anti-cancer therapy comprising an anti-clusterin antibody or antigen binding fragment thereof.

In some embodiments, the preparation of TILs or TILs culture is obtained from a subject that has been treated or is being treated with an anti-clusterin antibody or an antigen binding fragment thereof as a single agent or in combination therapy with a chemotherapeutic agent.

In some embodiments, the TILs are not genetically modified.

In other embodiments, the TILs are genetically modified.

In some embodiments, the preparation of TILs comprises TILs that are genetically modified.

In some embodiments, the preparation of TILs comprises TILs that express a chimeric antigen receptor.

In some embodiments, the preparation of TILs comprises TILs that express a transgenic T-cell receptor.

In some embodiments, the preparation of TILs is provided in an infusion bag.

In some embodiments, the preparation of tumor infiltrating lymphocytes (TILs) comprises a majority of CD45⁺ cells.

In some embodiments, the preparation of tumor infiltrating lymphocytes (TILs) comprises a majority of CD3⁺ cells.

In some embodiments, the preparation of tumor infiltrating lymphocytes (TILs) comprises a majority of CD4⁺ cells.

In some embodiments, the preparation of tumor infiltrating lymphocytes (TILs) comprises a majority of CD8⁺ cells.

In some embodiments, the preparation of tumor infiltrating lymphocytes (TILs) comprises a majority of cells that are CD4⁺ or CD8⁺ cells.

In some instances, the preparation of tumor infiltrating lymphocytes may comprise at least 50% of CD8+ lymphocytes. In other instances, the preparation of tumor infiltrating lymphocytes may comprise at least 60% of CD8+ lymphocytes. In yet other instances, the preparation of tumor infiltrating lymphocytes may comprise at least 70% of CD8+ lymphocytes. In additional instances, the preparation of tumor infiltrating lymphocytes may comprise at least 75% of CD8+ lymphocytes. In additional instances, the preparation of tumor infiltrating lymphocytes may comprise more than 75% of CD8+ lymphocytes. The preparation of tumor infiltrating lymphocytes may secrete intermediate to high levels of INFγ.

In an exemplary embodiment, the preparation of tumor infiltrating lymphocytes may be composed of tumor infiltrating lymphocytes cultures, each comprising at least 50% of CD8+ lymphocytes. In other exemplary embodiments, the preparation of tumor infiltrating lymphocytes may be composed of tumor infiltrating lymphocytes cultures each comprising at least 50% of CD8+ lymphocytes and secreting intermediate to high levels of INFγ. In other exemplary embodiments, the preparation of tumor infiltrating lymphocytes is composed of tumor infiltrating lymphocytes cultures each comprising at least 60% of CD8+ lymphocytes and secreting high levels of INFγ. In additional exemplary embodiments, the preparation of tumor infiltrating lymphocytes is composed of tumor infiltrating lymphocytes cultures each comprising at least 70% of CD8+ lymphocytes and secreting high levels of INFγ. In yet additional exemplary embodiments, the preparation of tumor infiltrating lymphocytes is composed of tumor infiltrating lymphocytes cultures each comprising at least 75% of CD8+ lymphocytes and secreting high levels of INFγ. In yet additional exemplary embodiments, the preparation of tumor infiltrating lymphocytes is composed of tumor infiltrating lymphocytes cultures each comprising more than 75% of CD8+ lymphocytes and secreting high levels of INFγ.

In some embodiments, each of the tumor infiltrating lymphocytes cultures may be obtained from the same tumor. In another embodiment, each of the tumor infiltrating lymphocytes cultures may be obtained from different tumors.

In other instances, the preparation of tumor infiltrating lymphocytes may comprise less than 10% of CD4+ lymphocytes. In yet other instances, the preparation of tumor infiltrating lymphocytes may comprise less than 7.5% of CD4+ lymphocytes. In other instances, the preparation of tumor infiltrating lymphocytes may comprise less than 5% of CD4+ lymphocytes. In other instances, the preparation of tumor infiltrating lymphocytes may comprise 2% of CD4+ lymphocytes or less.

The preparation of tumor infiltrating lymphocytes may be provided as an article of manufacture. Exemplary embodiments of article of manufacture include vials, flasks, syringes, infusion bags and the like.

### BRIEF DESCRIPTION OF THE DRAWINGS

FIG. 1: 4T1 Lung Metastases Are Immunologically “Cold” Which Prevents Immune Lymphocyte Infiltration. CD3+ and CD8+ T cells are present in the margins of 4T1 lung metastases resulting from the creation of a restrictive tumor microenvironment as a consequence of the epithelial to mesenchymal transitions that prevents lymphocytic infiltration.

FIG. 2A: Inhibition of EMT with the 16B5 Anti-sCLU mAb Results in B (B220) and T (CD3, CD4, CD8) Lymphocytes Infiltration in 4T1 Lung Metastases.

FIG. 2B-D: Picture of human tumor biopsies of patients treated with AB-16B5 as single agent.

FIG. 3: Graph of the number of metastatic lung nodules in 4T1-implanted animals treated with AB-16B5 in monotherapy or in combination with docetaxel.

FIG. 4A and FIG. 4B: 4T1 lung metastases from animals treated with AB-16B5 in monotherapy or in combination with docetaxel are infiltrated by B and T lymphocytes. 4T1 lung metastases were dissected at Day 36 post-implantation and processed with collagenase and hyaluronidase for immunophenotyping by flow cytometry.

Further scope, applicability and advantages of the present disclosure will become apparent from the non-restrictive detailed description given hereinafter. It should be understood, however, that this detailed description, while indicating exemplary embodiments of the disclosure, is given by way of example only, with reference to the accompanying drawings.

### DETAILED DESCRIPTION

### Definitions

Unless indicated otherwise, the amino acid numbering indicated for the dimerization domain are in accordance with the EU numbering system.

The use of the terms “a” and “an” and “the” and similar referents in the context of describing embodiments (especially in the context of the claims) are to be construed to cover both the singular and the plural, unless otherwise indicated herein or clearly contradicted by context.

Unless specifically stated or obvious from context, as used herein the term “or” is understood to be inclusive and covers both “or” and “and”.

The term “and/or” where used herein is to be taken as specific disclosure of each of the specified features or components with or without the other.

The terms “comprising”, “having”, “including”, and “containing” are to be construed as open-ended terms (i.e., meaning “including, but not limited to”) unless otherwise noted. The term “consisting of” is to be construed as close-ended.

The term “treatment” for purposes of this disclosure refers to both therapeutic treatment and prophylactic or preventative measures. Those in need of treatment include those already with the disorder as well as those prone to have the disorder or those in whom the disorder is to be prevented.

The term “EMT signature” as used herein refers to changes that are indicative of a loss of epithelial phenotype and/or acquisition of a mesenchymal phenotype that are observable at the cellular level and/or observable or measurable at the genetic level or protein level.

The term “about” or “approximately” with respect to a given value means that variation in the value is contemplated. In some embodiments, the term “about” or “approximately” shall generally mean a range within +/−20 percent, within +/−10 percent, within +/−5 percent, within +/−4 percent, within +/−3 percent, within +/−2 percent or within +/−1 percent of a given value or range.

The term “functional immune system” with respect to a subject means that the immune system of the subject is essentially not affected by cancer or by medication or that the subject is not immunosuppressed.

### Methods and Uses

Administration of an anti-cancer therapy comprising an anti-clusterin antibody or antigen binding fragment thereof promotes infiltration of tumor cells in the tumor microenvironment. Tumor infiltrating lymphocytes are isolated from the primary tumor or from tumor metastasis and expanded in vitro. Preparations of tumor infiltrating lymphocytes may be used in adoptive cell therapy.

The present disclosure therefore provides a method of treating a subject having cancer, which comprises a step of administering an anti-cancer therapy comprising an anti-clusterin antibody or antigen binding fragment thereof to the subject, a step of isolating and expanding tumor infiltrating lymphocytes (TILs) from the subject's tumor and a step of reinfusing a preparation of TILs to the subject.

The present disclosure also provides a method of treating cancer with tumor infiltrating lymphocytes (TILs) isolated and expanded from a tumor isolated from a subject treated with an anti-cancer therapy that comprises an anti-clusterin antibody or antigen binding fragment thereof.

TILs may thus be isolated from a subject that has received a prior treatment with at least an anti-clusterin antibody or antigen binding fragment thereof.

In some instances, the anti-cancer therapy is administered at least two weeks before isolation of TILs. In other instances, the anti-cancer therapy is administered at least three weeks before isolation of TILs. In yet other instances, the anti-cancer therapy is administered at least four weeks before isolation of TILs. In further instances, the anti-cancer therapy is administered at least five weeks before isolation of TILs. In yet further instances, the anti-cancer therapy is administered at least six weeks before isolation of TILs.

In some embodiments, the anti-cancer therapy is a combination therapy that comprises an anti-clusterin antibody or antigen binding fragment thereof and docetaxel. The anti-cancer therapy may be administered as a cycle of treatment that consist in administering the anti-clusterin antibody or antigen binding fragment thereof weekly and docetaxel once every three weeks.

In an exemplary embodiment, the anti-cancer therapy is administered for at least one cycle of treatment. In another exemplary embodiment, the anti-cancer therapy is administered for at least two cycles of treatment. In yet another exemplary embodiment, the anti-cancer therapy is administered for more than two cycles of treatment.

The method of the present disclosure may also comprise a step of administering an anti-cancer therapy that comprises an anti-clusterin antibody or antigen binding fragment thereof subsequent to the adoptive cell therapy.

In some embodiments, the anti-cancer therapy may be administered at least one week after the adoptive cell therapy. In another embodiment, the anti-cancer therapy may be administered at least two weeks after the adoptive cell therapy. In yet another embodiment, the anti-cancer therapy may be administered at least three weeks after the adoptive cell therapy. In further embodiments, the anti-cancer therapy may be administered at least four weeks after the adoptive cell therapy.

In some embodiments, the subsequent anti-cancer therapy is a combination therapy that comprises an anti-clusterin antibody or antigen binding fragment thereof and docetaxel. The subsequent anti-cancer therapy may be administered as a cycle of treatment that consist in administering the anti-clusterin antibody or antigen binding fragment thereof weekly and docetaxel once every three weeks.

In an exemplary embodiment, the subsequent anti-cancer therapy is administered for at least one cycle of treatment. In another exemplary embodiment, the subsequent anti-cancer therapy is administered for at least two cycles of treatment. In yet another exemplary embodiment, the subsequent anti-cancer therapy is administered for more than two cycles of treatment.

In some embodiments, the TILs may be obtained by a method known to a person skilled in the art.

In some embodiments, TILs are isolated and expanded by an in vitro or ex vivo method of generating tumor infiltrating lymphocytes.

TILs are usually processed by a method that comprises an initial culture phase and an expansion phase.

The initial culture phase may be carried out by placing tumor digests and/or tumor fragments in culture, typically in 24-well plates. During the initial culture phase, the TILs may become in suspension in cell culture media and the tumor cells may become adherent to the cell culture plate.

The tumor fragments may originate from a primary tumor or from tumor metastasis obtained from a subject treated with the anti-cancer therapy disclosed herein.

The initial culture phase involves culturing TILs in the presence of tumor cells. In some embodiments each fragment is cultured separately so as to obtain separate TILs cultures.

For the initial culture phase, the TILs culture may be supplied with cytokines. Exemplary embodiments of cytokines include IL-2 (recombinant human IL-2), IL-7 (recombinant human IL-7), IL-15 (recombinant human IL-15) and combination thereof.

The initial culture phase is typically carried out for a period ranging from two to five weeks. In some instances, the initial culture phase is carried out for least two weeks. In other instances, the initial culture phase is carried out for at least three weeks. In yet other instances, the initial culture phase is carried out for at least four weeks. In further instances, the initial culture phase is carried out for more than least four weeks.

Each TILs culture may be tested during or at the end of the initial culture phase so as to identify those having desirable anti-tumor activity. Alternatively, each TILs culture may be tested during or at the end of the initial culture phase so as to identify those having the highest proportion of lymphocytes. In some instances, T lymphocytes may be identified by cytometry using markers such as for example and without limitations, CD3, CD45 or combination thereof. In some instances, TILs culture having the highest proportion of cytotoxic lymphocytes may be selected.

The anti-tumor activity of a given TILs culture may be assessed for example, by the level of INFγ secreted in the presence of tumor cells. More particularly, an increase in INFγ secretion in the presence of tumor cells compared to baseline INFγ secretion may be indicative of the potential anti-tumor activity of a given TILs culture. In yet other instances, the anti-tumor activity of a given TILs culture may be determined by expression of activation markers. An exemplary embodiment of an activation marker is CD37. Expression of activation markers may be determined, for example, by cytometry. Other methods for testing anti-tumor activity may be used.

TILs that show anti-tumor activity are particularly contemplated for administration to the subject.

In some embodiments, TILs cultures showing evidence of INFγ secretion or an increase in INFγ secretion upon co-cultivation with tumor cells compared to baseline may be selected for the expansion phase.

In exemplary embodiments, INFγ secretion level of equal to or higher than 100 pg/ml is particularly contemplated for selection to the expansion phase.

In other exemplary embodiments, INFγ secretion level of equal to or higher than 300 pg/ml (intermediate level) is particularly contemplated for selection to the expansion phase.

In yet other exemplary embodiments, INFγ secretion level of equal to or higher than 500 pg/ml (high levels) is particularly contemplated for selection to the expansion phase.

In some embodiments, INFγ secretion is determined after at least two weeks in culture. In other embodiments, INFγ secretion is determined after at least three weeks in culture. In other embodiments, INFγ secretion is determined after at least four weeks in culture.

In some embodiments, TILs culture that have the highest proportion of cytotoxic T cells may be selected for the expansion phase or for administration to the subject. For example, TILs cultures having the highest proportion of CD8⁺ T lymphocytes are selected. In another example TILs cultures having at least 50% of CD8⁺ T lymphocytes are selected.

TILs culture having desirable characteristics may be pooled before or after the expansion phase or alternatively, individual TILs culture may be expanded.

In some embodiments, the expansion phase may involve removing tumor cells from the TILs culture or isolating immune cells from the culture. In some instances, CD8+ T-cells may be particularly selected from the culture for subsequent transfer to the subject.

For the expansion phase, if desired the TILs culture may also be supplied with cytokines. Exemplary embodiments of cytokines include IL-2 (recombinant human IL-2), IL-7 (recombinant human IL-7), IL-15 (recombinant human IL-15) and combination thereof. If desired, one or more cytokines may be excluded from the expansion phase.

The expansion phase is typically carried out for a period ranging from one to five weeks. In some instances, the expansion phase may be carried out for at least one week. In other instances, the expansion phase may be carried out for at least two weeks. In yet other instances, the expansion phase may be carried out for at least three weeks. In further instances, the expansion phase may be carried out for at least four weeks.

The preparation of TILs may be further tested for anti-tumor activity.

The method of the present disclosure may involve a step of processing TILs culture or TILs preparation so as to improve their characteristics. The processing may be carried out at one or more time point throughout the initial culture phase or throughout the expansion phase.

For example, the TILs culture or TILs preparation may be processed to remove components that may have a negative impact on the anti-tumor activity. In another example, the TILs culture or TILs preparation may be processed to remove components that may interfere with the growth or activity of cytotoxic lymphocytes.

In some exemplary embodiments, TILs culture or TILs preparation may be processed to remove TRegs.

In other exemplary embodiments, the TILs culture or TILs preparation may be processed to remove NKT cells.

In some embodiments, the method may include a step of removing tumor cells from the TILs culture or TILs preparation.

In some embodiments, the method may include a step of selecting CD45⁺ cells from the TILs culture or TILs preparation.

In some embodiments, the method may include a step of selecting CD4⁺ cells from the TILs culture or TILs preparation.

In some embodiments, the method may include a step of selecting CD8⁺ cells from the TILs culture or TILs preparation.

In some embodiments, the method may include a step of selecting tumor infiltrating lymphocytes cultures or TILs preparations that secrete INFγ at a level of equal to or higher than 100 pg/ml.

In some embodiments, the method may include a step of selecting tumor infiltrating lymphocytes cultures or TILs preparations that secrete INFγ at a level of equal to or higher than 300 pg/ml (intermediate level).

In some embodiments, the method may include a step of selecting tumor infiltrating lymphocytes cultures or TILs preparations that secrete INFγ at a level of equal to or higher than 500 pg/ml (high levels).

In some embodiments, the method may include a step of selecting tumor infiltrating lymphocytes cultures or TILs preparations that comprise at least 50% of CD8+ lymphocytes. In other embodiments, the method may include a step of selecting tumor infiltrating lymphocytes cultures or TILs preparations that comprise at least 60% of CD8+ lymphocytes. In yet other embodiments, the method may include a step of selecting tumor infiltrating lymphocytes cultures or TILs preparations that comprise at least 70% of CD8+ lymphocytes. In further embodiments, the method may include a step of selecting tumor infiltrating lymphocytes cultures or TILs preparations that comprise at least 75% of CD8+ lymphocytes. In additional embodiments, the method may include a step of selecting tumor infiltrating lymphocytes cultures or TILs preparations that comprise more than 75% of CD8+ lymphocytes.

In further embodiments, the method may include a step of selecting tumor infiltrating lymphocytes cultures or TILs preparations that comprise CD8+ lymphocytes and that secrete intermediate to high levels of INFγ.

Accordingly, in some instances, the method may comprise a step of selecting tumor infiltrating lymphocytes cultures or TILs preparations that comprise at least 50% of CD8+ lymphocytes and that secrete intermediate to high levels of INFγ. In other instances, the method may comprise a step of selecting tumor infiltrating lymphocytes cultures or TILs preparations that comprise at least 60% of CD8+ lymphocytes and that secrete intermediate to high levels. In yet other instances, the method may comprise a step of selecting tumor infiltrating lymphocytes cultures or TILs preparations that comprise at least 70% of CD8+ lymphocytes and that secrete intermediate to high levels. In additional instances, the method may comprise a step of selecting tumor infiltrating lymphocytes cultures or TILs preparations that comprise at least 75% of CD8+ lymphocytes and that secrete intermediate to high levels. In additional instances, the method may comprise a step of selecting tumor infiltrating lymphocytes cultures or TILs preparations that comprise more than 75% of CD8+ lymphocytes and that secrete intermediate to high levels.

In some embodiments, the method may comprise a step of pooling tumor infiltrating lymphocytes cultures or TILs preparations that comprise CD8+ lymphocytes and that secretes intermediate to high levels of INFγ.

Accordingly, in some embodiments, the method may comprise a step of pooling tumor infiltrating lymphocytes cultures each comprising at least 50% of CD8+ lymphocytes and secreting intermediate to high levels of INFγ. In other exemplary embodiments, the method may comprise a step of pooling tumor infiltrating lymphocytes cultures each comprising at least 60% of CD8+ lymphocytes and secreting intermediate to high levels of INFγ. In additional exemplary embodiments, the method may comprise a step of pooling tumor infiltrating lymphocytes cultures each comprising at least 70% of CD8+ lymphocytes and secreting intermediate to high levels of INFγ. In yet additional exemplary embodiments, the method may comprise a step of pooling tumor infiltrating lymphocytes cultures each comprising at least 75% of CD8+ lymphocytes and secreting intermediate to high levels of INFγ. In other exemplary embodiments, the method may comprise a step of pooling tumor infiltrating lymphocytes cultures each comprising more than 75% of CD8+ lymphocytes and secreting intermediate to high levels of INFγ.

In an exemplary embodiment, the method may comprise a step of selecting and/or pooling tumor infiltrating lymphocyte cultures that secrete intermediate levels of INFγ.

In an exemplary embodiment, the method may comprise a step of selecting and/or pooling tumor infiltrating lymphocyte cultures that secretes high levels of INFγ.

Similarly, preparations of TILs having diverse characteristics may be pooled.

In some embodiments, the preparation of TILs is obtained from a subject described herein.

In some embodiments, the preparation of TILs is obtained from a subject that has been treated or is treated with an anti-clusterin antibody or an antigen binding fragment thereof as a single agent.

In some embodiments, the preparation of TILs is obtained from a subject that has been treated or is treated with a combination therapy comprising an anti-clusterin antibody or an antigen binding fragment thereof and a chemotherapeutic agent.

In some embodiments, the chemotherapeutic agent is docetaxel.

In some embodiments, the tumor is resectable.

In some embodiments, the subject has a functional immune system.

In some embodiments, the TILs are obtained from a tumor or tumor fragments isolated by biopsy.

In accordance with the present disclosure, the in vitro or ex vivo method of generating tumor infiltrating lymphocytes comprises a step of contacting tumor fragments with an anti-clusterin antibody or antigen binding fragment thereof.

In accordance with the present disclosure, the anti-clusterin antibody or an antigen binding fragment thereof may be present and/or maintained during the initial culture phase of the method of generating tumor infiltrating lymphocytes.

In accordance with the present disclosure, the anti-clusterin antibody or an antigen binding fragment thereof may be present and/or maintained during the expansion phase of the method of generating tumor infiltrating lymphocytes.

The TILs may be genetically modified or not. For example, the TILs may express a chimeric antigen receptor. The basic structure of chimeric antigen receptors has been described in the literature (e.g., Gacerez, A. T. et al., J Cell Physiol. 231(12):2590-2598 (2016), Sadelain, M. et al. Cancer Discovery, 3(4):388-98, (2013), Zhang, C. et al., Biomarker Research, 5:22 (2017)). A chimeric antigen receptor usually comprises an extracellular antigen-binding domain typically in the form of a single chain Fv, a transmembrane domain, a costimulatory domain and an intracellular signaling domain.

In other examples, the TILs may express a transgenic T-cell receptor.

The TILs may be isolated from a primary tumor or from a tumor metastasis.

In accordance with the present disclosure, the anti-clusterin antibody or antigen binding fragment thereof may be administered at a dose and/or an administration interval and/or for a treatment period sufficient to result in infiltration of immune cells in the tumor microenvironment.

In accordance with the present disclosure, docetaxel may be administered at a dose and/or an administration interval and/or for a treatment period sufficient to allow chemotherapy-induced immunogenic modulation of tumor.

In some embodiments, the anti-clusterin antibody or antigen binding fragment thereof is as disclosed herein. For example, in some embodiments, the antic-clusterin antibody or antigen binding fragment thereof is humanized 16B5.

In some embodiments, the anti-clusterin antibody or antigen binding fragment thereof is administered prior to isolating the TILs. In some embodiments, the anti-clusterin antibody or antigen binding fragment thereof and chemotherapeutic agent are administered prior to isolating the TILs. In some embodiments, one or more treatment cycles are administered prior to isolating the TILs.

In some embodiments, the anti-clusterin antibody or antigen binding fragment thereof is administered after TILs are infused. In some embodiments, the anti-clusterin antibody or antigen binding fragment thereof and chemotherapeutic agent are administered after TILs are infused. In some embodiments, one or more treatment cycles are administered after TILs are infused.

In some embodiments, the preparation of TILs comprises CD3⁺ T cells.

In some embodiments, the preparation of TILs comprises CD4⁺ T cells.

In some embodiments, the preparation of TILs comprises CD8⁺ T cells.

In some embodiments, the preparation of TILs comprises B cells.

In some embodiments, the preparation of TILs comprises NK cells.

In some embodiments, the preparation of TILs comprises NK T cells.

In some embodiments, the preparation of TILs is selected for tumor antigen recognition.

In accordance with the present disclosure, the anti-clusterin antibody or antigen binding fragment thereof may be administered at a dosage, regimen and/or schedule disclosed herein.

In accordance with the present disclosure, docetaxel may be administered at a dosage, regimen and/or schedule disclosed herein.

In accordance with the present disclosure, the combination of anti-clusterin antibody or antigen binding fragment thereof and docetaxel may be administered at a dosage, regimen and/or schedule disclosed herein.

In accordance with the present disclosure the subject may have a carcinoma such as, for example, a metastatic carcinoma.

The present disclosure provides in yet another aspect thereof, a method of treating a subject having cancer which comprises administering tumor infiltrating lymphocytes (TILs) obtained by an in vitro or ex vivo method comprising a step of contacting the tumor fragments with an anti-clusterin antibody or antigen binding fragment thereof as a single agent or in combination therapy with a chemotherapeutic agent.

In some embodiments, the subject may have been previously treated with the anti-clusterin antibody or antigen binding fragment thereof or combination therapy.

In some embodiments, the subject has not been previously treated with the anti-clusterin antibody or antigen binding fragment thereof or combination therapy.

In accordance with the present disclosure, TILs are reinfused to the subject. TILs infusion protocols have been described in the literature. In some embodiments, the subject receives a lymphocyte-depleting preparative regimen prior to infusion of TILs. In some embodiments, the subject receives IL-2.

For example, Prior to infusion of the TIL product, patients may receive a non-myeloablative, lymphocyte-depleting preparative regimen consisting of cyclophosphamide (60 mg/kg/day×2 days intravenous) and fludarabine (25 mg/m²/day×5 days intravenous). Intravenous adoptive transfer of TILs may be followed by intravenous IL-2 (Proleukin) (600 000 IU/kg/dose every 8 hours up to tolerance or up to 15 doses).

### Preparation of TILs and TILs Culture

The present disclosure also provides a preparation of tumor infiltrating lymphocytes (TILs) obtained by the method described herein.

The present disclosure also provides a TILs culture obtained by the method described herein.

The expressions “preparation of TILs” and “TILs preparation” are used interchangeably.

Generally, the expression “preparation of TILs” is used to refer to a composition for administration in adoptive cell therapy. The expression “TILs culture” generally refers to a composition that is isolated, expanded or in the process of being isolated and/or expanded. A “TILs culture” may originate from a single cell clone or from a mixed population of cells. In some embodiments, a preparation of TILs may be composed of a single TILs culture or from several TILs cultures.

It is to be understood herein that “preparation of TILs” and “TILs culture” may have similar or identical characteristics. In some embodiments the preparation of TILs is a TILs culture.

In some embodiments, the preparation of TILs or TILs culture is obtained from a subject described herein.

In some embodiments, the preparation of TILs is a preparation of expanded TILs.

Accordingly, the present disclosure also provides a preparation of expanded tumor infiltrating lymphocytes (TILs) or TILs culture obtained by a method of treating a subject having cancer with an anti-clusterin antibody or antigen binding fragment thereof to the subject and isolating and expanding tumor infiltrating lymphocytes (TILs) from the subject's tumor.

In some embodiments, the preparation of TILs or TILs culture is obtained from a subject that has been treated or is being treated with an anti-clusterin antibody or an antigen binding fragment thereof as a single agent or in combination therapy with a chemotherapeutic agent.

In some embodiments, the TILs are not genetically modified.

In some embodiments, the TILs are genetically modified.

In some embodiments, the TILs express a chimeric antigen receptor.

In some embodiments, the TILs express a transgenic T-cell receptor.

In some embodiments, the TILs are provided in an infusion bag.

The preparation of tumor infiltrating lymphocytes or TILs culture may secrete intermediate to high levels of INFγ.

In exemplary embodiments, the preparation of tumor infiltrating lymphocytes or TILs culture secretes INFγ at a level of equal to or higher than 100 pg/ml.

In other exemplary embodiments, the preparation of tumor infiltrating lymphocytes or TILs culture secretes INFγ at a level of equal to or higher than 300 pg/ml (intermediate level).

In yet other exemplary embodiments, the preparation of tumor infiltrating lymphocytes or TILs culture secretes INFγ at a level of equal to or higher than 500 pg/ml (high levels).

In some embodiments, the preparation of tumor infiltrating lymphocytes (TILs) or TILs culture comprises a majority of CD45+ cells. For example, in some embodiments, the preparation of TILs or TILs culture may comprise at least 80% of CD45+ cells. For example, in other embodiments, the preparation of TILs or TILs culture may comprise at least 90% of CD45+ cells. In other embodiments, the preparation of TILs or TILs culture may comprise at least 95% of CD45+ cells. Yet in other embodiments, the preparation of TILs or TILs culture may comprise at least 99% of CD45+ cells. In other embodiments, the preparation of TILs or TILs culture may comprise only CD45+ cells.

In some embodiments, the preparation of tumor infiltrating lymphocytes (TILs) comprises a majority of CD4+ cells. For example, in some embodiments, the preparation of TILs or TILs culture may comprise more than 50% of CD4+ cells. In other embodiments, the preparation of TILs or TILs culture may comprise at least 60% of CD4+ cells. In yet other embodiments, the preparation of TILs or TILs culture may comprise at least 70% of CD4+ cells. In some embodiments, the preparation of TILs or TILs culture may comprise at least 80% of CD4+ cells. In additional embodiments, the preparation of TILs or TILs culture may comprise at least 90% of CD4+ cells. In other embodiments, the preparation of TILs or TILs culture may comprise at least 95% of CD4+ cells. Yet in other embodiments, the preparation of TILs or TILs culture may comprise at least 99% of CD4 cells. In other embodiments, the preparation of TILs or TILs culture may comprise only CD4 cells.

In some embodiments, the preparation of tumor infiltrating lymphocytes (TILs) or TILs culture comprises a majority of CD8 cells. In some instances, the preparation of tumor infiltrating lymphocytes or TILs culture may comprise at least 50% of CD8+ lymphocytes. For example, in some embodiments, the preparation of tumor infiltrating lymphocytes or TILs culture may comprise more than 50% of CD8⁺ cells. In other embodiments, the preparation of tumor infiltrating lymphocytes or TILs culture may comprise at least 60% of CD8⁺ cells. In yet other embodiments, the preparation of tumor infiltrating lymphocytes or TILs culture may comprise at least 70% of CD8⁺ cells. In yet other embodiments, the preparation of tumor infiltrating lymphocytes or TILs culture may comprise at least 75% of CD8⁺ cells. In some embodiments, the preparation of tumor infiltrating lymphocytes or TILs culture may comprise at least 80% of CD8 cells. In additional embodiments, the preparation of tumor infiltrating lymphocytes or TILs culture may comprise at least 90% of CD8 cells. In other embodiments, the preparation of tumor infiltrating lymphocytes or TILs culture may comprise at least 95% of CD8 cells. Yet in other embodiments, the preparation of tumor infiltrating lymphocytes or TILs culture may comprise at least 99% of CD8⁺ cells. In other embodiments, the preparation of tumor infiltrating lymphocytes or TILs culture may comprise only CD8⁺ cells. In some instances, the CD8+ cells are CD8+T lymphocytes.

In some embodiments, the preparation of tumor infiltrating lymphocytes or TILs culture may comprise CD8+ lymphocytes and may secrete intermediate to high levels of INFγ.

In some embodiments, the preparation of tumor infiltrating lymphocytes may be composed of tumor infiltrating lymphocytes cultures each comprising CD8+ lymphocytes and secreting intermediate to high levels of INFγ.

In an exemplary embodiment, the preparation of tumor infiltrating lymphocytes may be composed of tumor infiltrating lymphocytes cultures, each comprising at least 50% of CD8+ lymphocytes. In other exemplary embodiments, the preparation of tumor infiltrating lymphocytes may be composed of tumor infiltrating lymphocytes cultures each comprising at least 50% of CD8+ lymphocytes and secreting intermediate to high levels of INFγ. In other exemplary embodiments, the preparation of tumor infiltrating lymphocytes is composed of tumor infiltrating lymphocytes cultures each comprising at least 60% of CD8+ lymphocytes and secreting intermediate to high levels of INFγ. In additional exemplary embodiments, the preparation of tumor infiltrating lymphocytes is composed of tumor infiltrating lymphocytes cultures each comprising at least 70% of CD8+ lymphocytes and secreting intermediate to high levels of INFγ. In yet additional exemplary embodiments, the preparation of tumor infiltrating lymphocytes is composed of tumor infiltrating lymphocytes cultures each comprising at least 75% of CD8+ lymphocytes and secreting intermediate to high levels of INFγ. In vet additional exemplary embodiments, the preparation of tumor infiltrating lymphocytes is composed of tumor infiltrating lymphocytes cultures each comprising at least 80% of CD8+ lymphocytes and secreting intermediate to high levels of INFγ. In yet additional exemplary embodiments, the preparation of tumor infiltrating lymphocytes is composed of tumor infiltrating lymphocytes cultures each comprising at least 85% of CD8+ lymphocytes and secreting intermediate to high levels of INFγ. In vet additional exemplary embodiments, the preparation of tumor infiltrating lymphocytes is composed of tumor infiltrating lymphocytes cultures each comprising at least 90% of CD8+ lymphocytes and secreting intermediate to high levels of INFγ. In yet additional exemplary embodiments, the preparation of tumor infiltrating lymphocytes is composed of tumor infiltrating lymphocytes cultures each comprising at least 95% of CD8+ lymphocytes and secreting intermediate to high levels of INFγ.

In some embodiments, the preparation of tumor infiltrating lymphocytes (TILs) or TILs culture comprises a majority of cells that are CD4⁺ or CD8. For example, in some embodiments, the preparation of TILs or TILs culture may comprise more than 50% of cells that are CD4⁺ or CD8. In other embodiments, the preparation of TILs or TILs culture may comprise at least 60% of cells that are CD4 or CD8⁺. In vet other embodiments, the preparation of TILs or TILs culture may comprise at least 70% of cells that are CD4 or CD8⁺. In some embodiments, the preparation of TILs or TILs culture may comprise at least 80% of cells that are CD4⁺ or CD8⁺. In additional embodiments, the preparation of TILs or TILs culture may comprise at least 90% of cells that are CD4⁺ or CD8⁺. In other embodiments, the preparation of TILs or TILs culture may comprise at least 95% of cells that are CD4⁺ or CD8⁺. Yet in other embodiments, the preparation of TILs or TILs culture may comprise at least 99% of cells that are CD4⁺ or CD8⁺. In other embodiments, the preparation of TILs or TILs culture may comprise only cells that are CD4⁺ or CD8⁺.

In other instances, the preparation of tumor infiltrating lymphocytes or TILs culture may comprise less than 10% of CD4+ lymphocytes. In vet other instances, the preparation of tumor infiltrating lymphocytes or TILs culture may comprise less than 7.5% of CD4+ lymphocytes. In other instances, the preparation of tumor infiltrating lymphocytes or TILs culture may comprise less than 5% of CD4+ lymphocytes. In other instances, the preparation of tumor infiltrating lymphocytes or TILs culture may comprise 2% of CD4+ lymphocytes or less.

In exemplary embodiments, the preparation of TILs or TILs culture is characterized by an INFγ secretion level of equal to or higher than 100 pg/ml.

In another exemplary embodiment, the preparation of TILs or TILs culture is characterized by an INFγ secretion level of equal to or higher than 300 pg/ml (intermediate level).

In yet another exemplary embodiment, the preparation of TILs or TILs culture is characterized by an INFγ secretion level of equal to or higher than 500 pg/ml (high levels).

In some embodiments, the preparation of TILs as disclosed herein is administered to a subject in need. The preparation of TILs is autologous to the subject from which it was originally isolated.

In the method of the present disclosure, a preparation of TILs is infused to the subject. Typically, 108 to 10¹¹ cells are used for treating a subject. The subject may receive a lymphodepleting treatment prior to the adoptive cell therapy.

The subject may also receive high dose of IL-2. Exemplary embodiments of high dose of IL-2 includes 600,000 IU/kg or 720,000 IU/kg. The high dose of IL-2 may be provided by IV infusion every 8 h. The high dose of IL-2 may be provided for up to 15 consecutive doses. The consecutive doses may be provided, for example, over 5 days.

### Anti-Clusterin Antibodies or Antigen Binding Fragments Thereof.

In some embodiments, the anti-clusterin antibody or antigen binding fragment thereof of the present disclosure is capable of inhibiting epithelial to mesenchymal transition.

In some embodiments, the anti-clusterin antibody or antigen binding fragment thereof of the present disclosure is capable of binding to amino acids 421 and 443 of a C-terminal portion of a ß-subunit of human clusterin (SEQ ID NO: 41 see PCT/CA2006/001505 published under No. WO2007/030930 and international application No. PCT/CA2010/0001882 published under No. WO2011/063523 the entire content of which is incorporated herein by reference).

In some embodiments, the anti-clusterin antibody or antigen binding fragment thereof of the present disclosure is capable of binding to an epitope comprised within amino acids 421 and 443 of a C-terminal portion of a ß-subunit of human clusterin (SEQ ID NO: 41 see PCT/CA2006/001505 published under No. WO2007/030930 and international application No. PCT/CA2010/0001882 published under No. WO2011/063523 the entire content of which is incorporated herein by reference).

In some embodiments, the anti-clusterin antibody or antigen binding fragment thereof comprises the CDRs of an anti-clusterin antibody or antigen binding fragment thereof of the present disclosure.

In some embodiments, the anti-clusterin antibody or antigen binding fragment thereof is an antibody or antigen binding fragment thereof that is capable of competing with an anti-clusterin antibody or antigen binding fragment thereof of the present disclosure for the binding of clusterin (e.g., secreted clusterin (sCLU) or tumor-associated sCLU (TA-sCLU)) or for binding to a polypeptide comprising the amino acid sequence set forth in SEQ ID NO:41.

In some embodiments, the CDRs are identified using methods known to a person skilled in the art and which are reviewed in Antibody Engineering Vol. 2, Chapter 3 by Andrew C. R. Martin, the entire content of which is incorporated herein by reference.

In particular embodiments, all CDRs are identified using the Kabat definition which is the most commonly used definition (Wu and Kabat, 1970).

In particular embodiments, all CDRs are identified using the contact definition (MacCallum et al., 1996) which is likely to be the most useful for people wishing to perform mutagenesis to modify the affinity of an antibody since these are residues which take part in interactions with antigen.

In particular embodiments, the anti-clusterin antibody or antigen binding fragment thereof comprises a light chain variable region comprising the complementarity determining regions (CDRs) of the light chain variable region set forth in SEQ ID NO:9 and a heavy chain variable region comprising the CDRs of the heavy chain variable region set forth in SEQ ID NO:10.

In some exemplary embodiments, the anti-clusterin antibody or antigen binding fragment thereof comprises a light chain variable region comprising a CDRL1 having the amino acid sequence set forth in SEQ ID NO:1, a CDRL2 having the amino acid sequence set forth in SEQ ID NO:2, a CDRL3 having the amino acid sequence set forth in SEQ ID NO:3.

In some exemplary embodiments, the anti-clusterin antibody or antigen binding fragment thereof comprises a heavy chain variable region comprising a CDRH1 having the amino acid sequence set forth in SEQ ID NO:4, a CDRH2 having the amino acid sequence set forth in SEQ ID NO:5, a CDRH3 having the amino acid sequence set forth in SEQ ID NO:6.

In some exemplary embodiments, the anti-clusterin antibody or antigen binding fragment thereof comprises a heavy chain variable region comprising a CDRH1 having the amino acid sequence set forth in SEQ ID NO:35, a CDRH2 having the amino acid sequence set forth in SEQ ID NO:36, a CDRH3 having the amino acid sequence set forth in SEQ ID NO:37.

In some embodiments, the anti-clusterin antibody or antigen binding fragment thereof comprises a light chain variable region comprising a CDRL1 having the amino acid sequence set forth in SEQ ID NO:1, a CDRL2 having the amino acid sequence set forth in SEQ ID NO:2, a CDRL3 having the amino acid sequence set forth in SEQ ID NO:3 and a heavy chain variable region comprising a CDRH1 having the amino acid sequence set forth in SEQ ID NO:4, a CDRH2 having the amino acid sequence set forth in SEQ ID NO:5, a CDRH3 having the amino acid sequence set forth in SEQ ID NO:6.

In some embodiments, the anti-clusterin antibody or antigen binding fragment thereof comprises a light chain variable region comprising a CDRL1 having the amino acid sequence set forth in SEQ ID NO:1, a CDRL2 having the amino acid sequence set forth in SEQ ID NO:2, a CDRL3 having the amino acid sequence set forth in SEQ ID NO:3 and a heavy chain variable region comprising a CDRH1 having the amino acid sequence set forth in SEQ ID NO:35, a CDRH2 having the amino acid sequence set forth in SEQ ID NO:36, a CDRH3 having the amino acid sequence set forth in SEQ ID NO:37.

In some embodiments, the anti-clusterin antibody or antigen binding fragment thereof comprises a light chain variable region having an amino acid sequence having at least 80% identity with the amino acid sequence set forth in SEQ ID NO:7 and a heavy chain variable region having an amino acid sequence at least 80% identity with the amino acid sequence set forth in SEQ ID NO:8.

In some embodiments, the anti-clusterin antibody or antigen binding fragment thereof comprises a light chain variable region having an amino acid sequence having at least 90% identity with the amino acid sequence set forth in SEQ ID NO:7 and a heavy chain variable region having an amino acid sequence at least 90% identity with the amino acid sequence set forth in SEQ ID NO:8.

In some embodiments, the anti-clusterin antibody or antigen binding fragment thereof comprises a light chain variable region having an amino acid sequence identical to the amino acid sequence set forth in SEQ ID NO:7 and a heavy chain variable region having an amino acid sequence identical to the amino acid sequence set forth in SEQ ID NO:8.

In some embodiments, the anti-clusterin antibody or antigen binding fragment thereof is capable of competing with an antibody comprising a light chain variable region having the amino acid sequence set forth in SEQ ID NO:7 and a heavy chain variable region having the amino acid sequence set forth in SEQ ID NO:8 for the binding of clusterin (e.g., secreted clusterin (sCLU) or tumor-associated sCLU (TA-sCLU)) or for binding to a polypeptide comprising the amino acid sequence set forth in SEQ ID NO:41.

In some embodiments, the anti-clusterin antibody or antigen binding fragment thereof comprises a light chain variable region having an amino acid sequence having at least 80% identity with the amino acid sequence set forth in SEQ ID NO:9 and a heavy chain variable region having an amino acid sequence at least 80% identity with the amino acid sequence set forth in SEQ ID NO: 10.

In some embodiments, the anti-clusterin antibody or antigen binding fragment thereof comprises a light chain variable region having an amino acid sequence having at least 90% identity with the amino acid sequence set forth in SEQ ID NO:9 and a heavy chain variable region having an amino acid sequence at least 90% identity with the amino acid sequence set forth in SEQ ID NO:10.

In some embodiments, the anti-clusterin antibody or antigen binding fragment thereof comprises a light chain variable region having an amino acid sequence identical to the amino acid sequence set forth in SEQ ID NO:9 and a heavy chain variable region having an amino acid sequence identical to the amino acid sequence set forth in SEQ ID NO:10.

In some embodiments, the anti-clusterin antibody or antigen binding fragment thereof is capable of competing with an antibody comprising a light chain variable region having the amino acid sequence set forth in SEQ ID NO:9 and a heavy chain variable region having the amino acid sequence set forth in SEQ ID NO:10 for the binding of clusterin (e.g., secreted clusterin (sCLU) or tumor-associated sCLU (TA-sCLU)) or for binding to a polypeptide comprising the amino acid sequence set forth in SEQ ID NO:41.

In some embodiments, the anti-clusterin antibody or antigen binding fragment thereof comprises a light chain having an amino acid sequence having at least 80% identity with the amino acid sequence set forth in SEQ ID NO:11 and a heavy chain having an amino acid sequence having at least 80% identity with the amino acid sequence set forth in SEQ ID NO:12.

In some embodiments, the anti-clusterin antibody or antigen binding fragment thereof comprises a light chain having an amino acid sequence having at least 90% identity with the amino acid sequence set forth in SEQ ID NO:11 and a heavy chain having an amino acid sequence having at least 90% identity with the amino acid sequence set forth in SEQ ID NO:12.

In some embodiments, the anti-clusterin antibody or antigen binding fragment thereof comprises a light chain having an amino acid sequence identical the amino acid sequence set forth in SEQ ID NO:11 and a heavy chain having an amino acid sequence identical to the amino acid sequence set forth in SEQ ID NO:12.

In some embodiments, the anti-clusterin antibody or antigen binding fragment thereof is capable of competing with an antibody comprising a light chain having the amino acid sequence set forth in SEQ ID NO:11 and a heavy chain having the amino acid sequence set forth in SEQ ID NO:12 for the binding of clusterin (e.g., secreted clusterin (sCLU) or tumor-associated sCLU (TA-sCLU)) or for binding to a polypeptide comprising the amino acid sequence set forth in SEQ ID NO:41.

In other particular embodiments, the anti-clusterin antibody or antigen binding fragment thereof comprises a light chain variable region comprising a CDRL1 having the amino acid sequence set forth in SEQ ID NO:15, a CDRL2 having the amino acid sequence set forth in SEQ ID NO:16, a CDRL3 having the amino acid sequence set forth in SEQ ID NO:17.

In some exemplary embodiments, the anti-clusterin antibody or antigen binding fragment thereof comprises a heavy chain variable region comprising a CDRH1 having the amino acid sequence set forth in SEQ ID NO:18, a CDRH2 having the amino acid sequence set forth in SEQ ID NO:19, a CDRH3 having the amino acid sequence set forth in SEQ ID NO:20.

In some exemplary embodiments, the anti-clusterin antibody or antigen binding fragment thereof comprises a heavy chain variable region comprising a CDRH1 having the amino acid sequence set forth in SEQ ID NO:38, a CDRH2 having the amino acid sequence set forth in SEQ ID NO:39, a CDRH3 having the amino acid sequence set forth in SEQ ID NO:40.

In some embodiments, the anti-clusterin antibody or antigen binding fragment thereof comprises a light chain variable region comprising a CDRL1 having the amino acid sequence set forth in SEQ ID NO:15, a CDRL2 having the amino acid sequence set forth in SEQ ID NO:16, a CDRL3 having the amino acid sequence set forth in SEQ ID NO:17 and a heavy chain variable region comprising a CDRH1 having the amino acid sequence set forth in SEQ ID NO:18, a CDRH2 having the amino acid sequence set forth in SEQ ID NO:19, a CDRH3 having the amino acid sequence set forth in SEQ ID NO:20.

In some embodiments, the anti-clusterin antibody or antigen binding fragment thereof comprises a light chain variable region comprising a CDRL1 having the amino acid sequence set forth in SEQ ID NO:15, a CDRL2 having the amino acid sequence set forth in SEQ ID NO:16, a CDRL3 having the amino acid sequence set forth in SEQ ID NO:17 and a heavy chain variable region comprising a CDRH1 having the amino acid sequence set forth in SEQ ID NO:38, a CDRH2 having the amino acid sequence set forth in SEQ ID NO:39, a CDRH3 having the amino acid sequence set forth in SEQ ID NO:40.

In some embodiments, the anti-clusterin antibody or antigen binding fragment thereof comprises a light chain variable region having an amino acid sequence having at least 80% identity with the amino acid sequence set forth in SEQ ID NO:21 and a heavy chain variable region having an amino acid sequence at least 80% identity with the amino acid sequence set forth in SEQ ID NO:22.

In some embodiments, the anti-clusterin antibody or antigen binding fragment thereof comprises a light chain variable region having an amino acid sequence having at least 90% identity with the amino acid sequence set forth in SEQ ID NO:21 and a heavy chain variable region having an amino acid sequence at least 90% identity with the amino acid sequence set forth in SEQ ID NO:22.

In some embodiments, the anti-clusterin antibody or antigen binding fragment thereof comprises a light chain variable region having an amino acid sequence identical to the amino acid sequence set forth in SEQ ID NO:21 and a heavy chain variable region having an amino acid sequence identical to the amino acid sequence set forth in SEQ ID NO:22.

In some embodiments, the anti-clusterin antibody or antigen binding fragment thereof is capable of competing with an antibody comprising a light chain variable region having the amino acid sequence set forth in SEQ ID NO:21 and a heavy chain variable region having the amino acid sequence set forth in SEQ ID NO:22 for the binding of clusterin (e.g., secreted clusterin (sCLU) or tumor-associated sCLU (TA-sCLU)) or for binding to a polypeptide comprising the amino acid sequence set forth in SEQ ID NO:41.

In some embodiments, the anti-clusterin antibody or antigen binding fragment thereof comprises a light chain variable region having an amino acid sequence having at least 80% identity with the amino acid sequence set forth in SEQ ID NO:23 and a heavy chain variable region having an amino acid sequence at least 80% identity with the amino acid sequence set forth in SEQ ID NO:24.

In some embodiments, the anti-clusterin antibody or antigen binding fragment thereof comprises a light chain variable region having an amino acid sequence having at least 90% identity with the amino acid sequence set forth in SEQ ID NO:23 and a heavy chain variable region having an amino acid sequence at least 90% identity with the amino acid sequence set forth in SEQ ID NO:24.

In some embodiments, the anti-clusterin antibody or antigen binding fragment thereof comprises a light chain variable region having an amino acid sequence identical to the amino acid sequence set forth in SEQ ID NO:23 and a heavy chain variable region having an amino acid sequence identical to the amino acid sequence set forth in SEQ ID NO:24.

In some embodiments, the anti-clusterin antibody or antigen binding fragment thereof is capable of competing with an antibody comprising a light chain variable region having the amino acid sequence set forth in SEQ ID NO:23 and a heavy chain variable region having the amino acid sequence set forth in SEQ ID NO:24 for the binding of clusterin (e.g., secreted clusterin (sCLU) or tumor-associated sCLU (TA-sCLU)) or for binding to a polypeptide comprising the amino acid sequence set forth in SEQ ID NO:41.

In some embodiments, the anti-clusterin antibody or antigen binding fragment thereof comprises a light chain having an amino acid sequence having at least 80% identity with the amino acid sequence set forth in SEQ ID NO:25 and a heavy chain having an amino acid sequence having at least 80% identity with the amino acid sequence set forth in SEQ ID NO:26.

In some embodiments, the anti-clusterin antibody or antigen binding fragment thereof comprises a light chain having an amino acid sequence having at least 90% identity with the amino acid sequence set forth in SEQ ID NO:25 and a heavy chain having an amino acid sequence having at least 90% identity with the amino acid sequence set forth in SEQ ID NO:26.

In some embodiments, the anti-clusterin antibody or antigen binding fragment thereof comprises a light chain having an amino acid sequence identical the amino acid sequence set forth in SEQ ID NO:25 and a heavy chain having an amino acid sequence identical to the amino acid sequence set forth in SEQ ID NO:26.

In some embodiments, the anti-clusterin antibody or antigen binding fragment thereof is capable of competing with an antibody comprising a light chain having the amino acid sequence set forth in SEQ ID NO:25 and a heavy chain having the amino acid sequence set forth in SEQ ID NO:26 for the binding of clusterin (e.g., secreted clusterin (sCLU) or tumor-associated sCLU (TA-sCLU)) or for binding to a polypeptide comprising the amino acid sequence set forth in SEQ ID NO:41.

In yet other particular embodiments, the anti-clusterin antibody or antigen binding fragment thereof comprises the CDRs, variable regions or full chains amino acid sequence of the antibody or antigen binding fragment thereof listed in Table 5. The amino acid sequence of antibodies identified as 16B5, 21B12, 20E11, 11E2 and 16C11 is disclosed in international application No. PCT/CA2006/001505 filed on Sep. 13, 2006 and published on Mar. 22, 2007 under no. WO2007/030930 the entire content of which is incorporated herein by reference. The amino acid sequence of murine 16B5, humanized 16B5, murine 21B12 and humanized 21B12 is disclosed in international application No. PCT/CA2010/001882 filed on Nov. 24, 2010 and published on Jun. 3, 2011 under No. WO2011/063523, the entire content of which is incorporated herein by reference.

In yet further particular embodiments, the anti-clusterin antibody or antigen binding fragment thereof may be able to compete with one or more of the antibody or antigen binding fragment thereof listed in Table 5.

### Subject

In some aspects and embodiments of the present disclosure, the subject is a human subject.

In some aspects and embodiments of the present disclosure, the subject is a subject having cancer.

In other aspects and embodiments of the present disclosure, the subject is a subject having cancer and having a functional immune system.

In some embodiments, the subject has a carcinoma.

In some embodiments, the subject has an endometrial cancer, a breast cancer, a liver cancer, a prostate cancer, a renal cancer, a bladder cancer, a cervical cancer, an ovarian cancer, a colorectal cancer, a pancreatic cancer, a lung cancer, a gastric cancer, a head and neck cancer, a thyroid cancer, a cholangiocarcinoma, a mesothelioma or a melanoma.

In some embodiments, the subject has a metastatic carcinoma.

In some embodiments, the subject has a metastatic endometrial cancer, a metastatic breast cancer, a metastatic liver cancer, a metastatic prostate cancer, a metastatic renal cancer, a metastatic bladder cancer, a metastatic cervical cancer, a metastatic ovarian cancer, a metastatic colorectal cancer, a metastatic pancreatic cancer, a metastatic lung cancer, a metastatic gastric cancer, a metastatic head and neck cancer, a metastatic thyroid cancer, a metastatic cholangiocarcinoma, a metastatic mesothelioma or a metastatic melanoma.

In some embodiments, the subject has non-small cell lung cancer (NSCLC).

In some embodiments, the subject has metastatic NSCLC.

In some embodiments, the subject has stage III to IV NSCLC.

In some embodiments, the subject has breast cancer.

In some embodiments, the subject has metastatic breast cancer.

In some embodiments, the subject has prostate cancer.

In some embodiments, the subject has metastatic prostate cancer.

In some embodiments, the subject has gastric cancer.

In some embodiments, the subject has metastatic gastric cancer.

In some embodiments, the subject has head and neck cancer.

In some embodiments, the subject has metastatic head and neck cancer.

In some embodiments, the subject has thyroid cancer.

In some embodiments, the subject has metastatic thyroid cancer.

In some embodiments, the subject has ovarian cancer.

In some embodiments, the subject has metastatic ovarian cancer.

In some embodiments, the subject has endometrial cancer.

In some embodiments, the subject has metastatic endometrial cancer.

In some embodiments, the subject has liver cancer.

In some embodiments, the subject has metastatic liver cancer.

In some embodiments, the subject has colorectal cancer.

In some embodiments, the subject has metastatic colorectal cancer.

In some embodiments, the subject has pancreatic cancer.

In some embodiments, the subject has metastatic pancreatic cancer.

In some embodiments, the subject has cholangiocarcinoma.

In some embodiments, the subject has metastatic cholangiocarcinoma.

In some embodiments, the subject has mesothelioma.

In some embodiments, the subject has metastatic mesothelioma.

In some embodiments, the subject has melanoma.

In some embodiments, the subject has metastatic melanoma.

In some embodiments, the subject has or is selected for having a tumor characterized as immunologically cold.

In some embodiments, the subject has or is selected for having a tumor characterized as immunologically warm or hot that is non-responsive to immunotherapy.

In some embodiments, the subject has or is selected for having a tumor showing sign of an epithelial to mesenchymal transition (EMT) signature.

As used herein the term “tumor” refers to the primary tumor or to tumor metastases or lesions.

In some embodiments, the subject has or is selected for having a carcinoma that progressed after a first line immune checkpoint therapy.

In some embodiments, the subject has or is selected for having a carcinoma that has failed prior treatment with an immune checkpoint therapy and platinum-containing doublet treatment.

In some embodiments, the subject has or is selected for having a carcinoma that has failed prior treatment with an immune checkpoint therapy and a platinum-containing doublet treatment administered simultaneously or sequentially.

In some embodiments, the subject has or is selected for having a carcinoma that has failed prior treatment with an anti-PD1 or PDL-1 immune checkpoint antibody and a platinum-containing doublet treatment.

In some embodiments, the subject has or is selected for having a carcinoma that has failed prior treatment with ipilimumab, nivolumab, pembrolizumab, cemiplimab, atezolizumab, avelumab, or durvalumab and a platinum-containing doublet treatment.

In some embodiments, the subject has or is selected for having a carcinoma that has failed prior treatment with an anti-PD1 or PDL-1 immune checkpoint antibody and a platinum-containing doublet treatment simultaneously or sequentially.

In some embodiments, the subject is not immunosuppressed.

In some embodiments, the subject has not received an immunosuppressive medication within 14 days, 7 days, 6 days, 5 days, 4 days, 3 days, 2 days or 1 day prior to treatment. In some embodiments, the subject may have received corticosteroids prior to treatment.

In some embodiments, the subject has not received prior treatment with docetaxel.

In some embodiments, the subject is treated for at least two cycles of treatment.

In some embodiments, the subject receives lymphocyte-depleting preparative regimen prior to infusion of TILs.

### Dosages, Treatment Regimens and Schedules

In accordance with an aspect of the present disclosure, the subject is treated with an anti-cancer therapy that comprises an anti-clusterin antibody or an antigen binding fragment thereof prior to isolation of tumor infiltrating lymphocytes.

Accordingly, the anti-clusterin antibody or antigen binding fragment thereof is therefore administered at a dose sufficient to result in infiltration of immune cells in the tumor microenvironment.

In some embodiments, the dose of the anti-clusterin antibody or antigen binding fragment thereof is a therapeutically effective and safe dose.

In accordance with the present disclosure, the anti-clusterin antibody or antigen binding fragment thereof is administered at an administration interval sufficient to result in infiltration of immune cells in the tumor microenvironment.

In accordance with the present disclosure, the anti-clusterin antibody or antigen binding fragment thereof is administered for a treatment period sufficient to result in infiltration of immune cells in the tumor microenvironment.

In some embodiments, the anti-clusterin antibody or antigen binding fragment thereof is administered at a dose, administration interval and/or treatment period sufficient to result in infiltration of immune cells in the tumor microenvironment.

In accordance with another aspect of the present disclosure, the subject is treated with a combination therapy comprising an anti-clusterin antibody or an antigen binding fragment thereof and docetaxel prior to isolation of tumor infiltrating lymphocytes.

In some embodiments, the dose of docetaxel is a therapeutically effective and safe dose.

In accordance with the present disclosure, docetaxel is administered at an administration interval sufficient to allow chemotherapy-induced immunogenic modulation of tumor.

In accordance with the present disclosure, docetaxel is administered for a treatment period sufficient to allow chemotherapy-induced immunogenic modulation of tumor.

In some embodiments, docetaxel is administered at a dose and/or an administration interval and/or for a treatment period sufficient to allow chemotherapy-induced immunogenic modulation of tumor.

Accordingly, the anti-clusterin antibody or antigen binding fragment thereof and docetaxel are therefore administered at a dose sufficient to result in infiltration of immune cells in the tumor microenvironment and/or to allow chemotherapy-induced immunogenic modulation of tumor.

In accordance with yet another aspect of the present disclosure, the subject is treated with an anti-cancer therapy that comprises an anti-clusterin antibody or an antigen binding fragment thereof after reinfusion of tumor infiltrating lymphocytes.

In accordance with a further aspect of the present disclosure, the subject is treated with a combination therapy comprising an anti-clusterin antibody or an antigen binding fragment thereof and docetaxel after reinfusion of tumor infiltrating lymphocytes.

In accordance with an exemplary embodiment of the disclosure, the anti-clusterin antibody or antigen binding fragment thereof is administered once weekly.

In accordance with another exemplary embodiment of the disclosure, the anti-clusterin antibody or antigen binding fragment thereof is administered twice weekly.

In accordance with yet another exemplary embodiment of the disclosure, the anti-clusterin antibody or antigen binding fragment thereof is administered thrice weekly.

In accordance with a further exemplary embodiment of the disclosure, the anti-clusterin antibody or antigen binding fragment thereof is administered once every two weeks.

In accordance with yet a further exemplary embodiment of the disclosure, the anti-clusterin antibody or antigen binding fragment thereof is administered once every three weeks.

In accordance with an additional exemplary embodiment of the disclosure, the anti-clusterin antibody or antigen binding fragment thereof is administered once every four weeks.

In some embodiments, the anti-clusterin antibody or antigen binding fragment thereof is administered weekly for a period of at least two weeks before isolation of TILs. In other embodiments, the anti-clusterin antibody or antigen binding fragment thereof is administered weekly for a period of at least three weeks before isolation of TILs. In yet other embodiments, the anti-clusterin antibody or antigen binding fragment thereof is administered weekly for a period of at least four weeks before isolation of TILs. In further embodiments, the anti-clusterin antibody or antigen binding fragment thereof is administered weekly for a period of at least five weeks before isolation of TILs. In yet further embodiments, the anti-clusterin antibody or antigen binding fragment thereof is administered weekly for a period of at least six weeks before isolation of TILs. In accordance with the present disclosure, the anti-clusterin antibody or antigen binding fragment thereof is administered at a dose of between approximately 3 mg/kg and approximately 20 mg/kg.

In some embodiments, the anti-clusterin antibody or antigen binding fragment thereof is administered at a dose of approximately 3.0 mg/kg.

In some embodiments, the anti-clusterin antibody or antigen binding fragment thereof is administered at a dose of approximately 4.0 mg/kg.

In some embodiments, the anti-clusterin antibody or antigen binding fragment thereof is administered at a dose of approximately 5.0 mg/kg.

In some embodiments, the anti-clusterin antibody or antigen binding fragment thereof is administered at a dose of approximately 6.0 mg/kg.

In some embodiments, the anti-clusterin antibody or antigen binding fragment thereof is administered at a dose of approximately 7.0 mg/kg.

In some embodiments, the anti-clusterin antibody or antigen binding fragment thereof is administered at a dose of approximately 8.0 mg/kg.

In some embodiments, the anti-clusterin antibody or antigen binding fragment thereof is administered at a dose of approximately 9.0 mg/kg.

In some embodiments, the anti-clusterin antibody or antigen binding fragment thereof is administered at a dose of approximately 10.0 mg/kg.

In some embodiments, the anti-clusterin antibody or antigen binding fragment thereof is administered at a dose of approximately 11.0 mg/kg.

In some embodiments, the anti-clusterin antibody or antigen binding fragment thereof is administered at a dose of approximately 12.0 mg/kg.

In some embodiments, the anti-clusterin antibody or antigen binding fragment thereof is administered at a dose of approximately 13.0 mg/kg.

In some embodiments, the anti-clusterin antibody or antigen binding fragment thereof is administered at a dose of approximately 14.0 mg/kg.

In some embodiments, the anti-clusterin antibody or antigen binding fragment thereof is administered at a dose of approximately 15.0 mg/kg.

In some embodiments, the anti-clusterin antibody or antigen binding fragment thereof is administered at a dose of approximately 16.0 mg/kg.

In some embodiments, the anti-clusterin antibody or antigen binding fragment thereof is administered at a dose of approximately 17.0 mg/kg.

In some embodiments, the anti-clusterin antibody or antigen binding fragment thereof is administered at a dose of approximately 18.0 mg/kg.

In some embodiments, the anti-clusterin antibody or antigen binding fragment thereof is administered at a dose of approximately 19.0 mg/kg.

In some embodiments, the anti-clusterin antibody or antigen binding fragment thereof is administered at a dose of approximately 20.0 mg/kg.

In accordance with the present disclosure, the anti-clusterin antibody or antigen binding fragment thereof is humanized 16B5 and is administered at a dose of between approximately 3 mg/kg and approximately 20 mg/kg.

In accordance with the present disclosure, humanized 16B5 is administered at a dose of between approximately 4 mg/kg and approximately 20 mg/kg.

In accordance with the present disclosure, the humanized 16B5 is administered at a dose of between approximately 5 mg/kg and approximately 20 mg/kg.

In accordance with the present disclosure, humanized 16B5 is administered at a dose of between approximately 6 mg/kg and approximately 20 mg/kg.

In accordance with the present disclosure, humanized 16B5 is administered at a dose of between approximately 6 mg/kg and approximately 18 mg/kg.

In accordance with the present disclosure, humanized 16B5 is administered at a dose of between approximately 6 mg/kg and approximately 17 mg/kg.

In accordance with the present disclosure, humanized 16B5 is administered at a dose of between approximately 6 mg/kg and approximately 16 mg/kg.

In accordance with the present disclosure, humanized 16B5 administered at a dose of between approximately 6 mg/kg and approximately 15 mg/kg.

In accordance with the present disclosure, humanized 16B5 is administered at a dose of between approximately 6 mg/kg and approximately 14 mg/kg.

In accordance with the present disclosure, humanized 16B5 is administered at a dose of between approximately 6 mg/kg and approximately 13 mg/kg.

In accordance with the present disclosure, humanized 16B5 is administered at a dose of between approximately 6 mg/kg and approximately 12 mg/kg

In accordance with the present disclosure, humanized 16B5 is administered at a dose of between approximately 7 mg/kg and approximately 12 mg/kg.

In accordance with the present disclosure, humanized 16B5 is administered at a dose of between approximately 8 mg/kg and approximately 12 mg/kg.

In accordance with the present disclosure, humanized 16B5 is administered at a dose of between approximately 9 mg/kg and approximately 12 mg/kg.

In some embodiments, the anti-clusterin antibody or antigen binding fragment thereof is humanized 16B5 and is administered at a dose of approximately 3.0 mg/kg.

In some embodiments, the anti-clusterin antibody or antigen binding fragment thereof is humanized 16B5 and is administered at a dose of approximately 4.0 mg/kg.

In some embodiments, the anti-clusterin antibody or antigen binding fragment thereof is humanized 16B5 and is administered at a dose of approximately 5.0 mg/kg.

In some embodiments, the anti-clusterin antibody or antigen binding fragment thereof is humanized 16B5 and is administered at a dose of approximately 6.0 mg/kg.

In some embodiments, the anti-clusterin antibody or antigen binding fragment thereof is humanized 16B5 and is administered at a dose of approximately 7.0 mg/kg.

In some embodiments, the anti-clusterin antibody or antigen binding fragment thereof is humanized 16B5 and is administered at a dose of approximately 8.0 mg/kg.

In some embodiments, the anti-clusterin antibody or antigen binding fragment thereof is humanized 16B5 and is administered at a dose of approximately 9.0 mg/kg.

In some embodiments, the anti-clusterin antibody or antigen binding fragment thereof is humanized 16B5 and is administered at a dose of approximately 10.0 mg/kg.

In some embodiments, the anti-clusterin antibody or antigen binding fragment thereof is humanized 16B5 and is administered at a dose of approximately 11.0 mg/kg.

In some embodiments, the anti-clusterin antibody or antigen binding fragment thereof is humanized 16B5 and is administered at a dose of approximately 12.0 mg/kg.

In some embodiments, the anti-clusterin antibody or antigen binding fragment thereof is humanized 16B5 and is administered at a dose of approximately 13.0 mg/kg.

In some embodiments, the anti-clusterin antibody or antigen binding fragment thereof is humanized 16B5 and is administered at a dose of approximately 14.0 mg/kg.

In some embodiments, the anti-clusterin antibody or antigen binding fragment thereof is humanized 16B5 and is administered at a dose of approximately 15.0 mg/kg.

In some embodiments, the anti-clusterin antibody or antigen binding fragment thereof is humanized 16B5 and is administered at a dose of approximately 16.0 mg/kg.

In some embodiments, the anti-clusterin antibody or antigen binding fragment thereof is humanized 16B5 and is administered at a dose of approximately 17.0 mg/kg.

In some embodiments, the anti-clusterin antibody or antigen binding fragment thereof is humanized 16B5 and is administered at a dose of approximately 18.0 mg/kg.

In some embodiments, the anti-clusterin antibody or antigen binding fragment thereof is humanized 16B5 and is administered at a dose of approximately 19.0 mg/kg.

In some embodiments, the anti-clusterin antibody or antigen binding fragment thereof is humanized 16B5 and is administered at a dose of approximately 20.0 mg/kg.

In accordance with an exemplary embodiment of the disclosure, docetaxel is administered once every week.

In accordance with another exemplary embodiment of the disclosure, docetaxel is administered once every two weeks.

In accordance with yet another exemplary embodiment of the disclosure, docetaxel is administered once every three weeks.

In accordance with a further exemplary embodiment of the disclosure, docetaxel is administered once every four weeks.

In accordance with a further exemplary embodiment of the disclosure, docetaxel is administered once every five weeks.

In accordance with a further exemplary embodiment of the disclosure, docetaxel is administered once every six weeks.

In accordance with the present disclosure, docetaxel is administered at a dose of between approximately 60 mg/m² to approximately 100 mg/m².

In accordance with the present disclosure docetaxel is administered at a dose of between approximately 60 mg/m² to approximately 95 mg/m².

In accordance with the present disclosure docetaxel is administered at a dose of between approximately 60 mg/m² to approximately 90 mg/m².

In accordance with the present disclosure docetaxel is administered at a dose of between approximately 60 mg/m² to approximately 85 mg/m².

In accordance with the present disclosure docetaxel is administered at a dose of between approximately 60 mg/m² to approximately 80 mg/m².

In accordance with the present disclosure docetaxel is administered at a dose of between approximately 60 mg/m² to approximately 75 mg/m².

In accordance with the present disclosure docetaxel is administered at a dose of between approximately 70 mg/m² to approximately 75 mg/m².

In some embodiments, docetaxel is administered at a dose of approximately 60 mg/m².

In some embodiments, docetaxel is administered at a dose of approximately 65 mg/m².

In some embodiments, docetaxel is administered at a dose of approximately 70 mg/m².

In some embodiments, docetaxel is administered at a dose of approximately 75 mg/m².

In some embodiments, docetaxel is administered at a dose of approximately 80 mg/m².

In some embodiments, docetaxel is administered at a dose of approximately 85 mg/m².

In some embodiments, docetaxel is administered at a dose of approximately 90 mg/m².

In some embodiments, docetaxel is administered at a dose of approximately 95 mg/m².

In some embodiments, docetaxel is administered at a dose of approximately 100 mg/m².

In some embodiments, the anti-clusterin antibody or antigen binding fragment thereof is administered at a dose of approximately 12 mg/kg once weekly, and docetaxel is administered at a dose of approximately 75 mg/m² once every three weeks.

In some embodiments, the anti-clusterin antibody or antigen binding fragment thereof is administered at a dose of approximately 12 mg/kg once weekly, and docetaxel is administered at a dose of approximately 60 mg/m² once every three weeks.

In some embodiments, the anti-clusterin antibody or antigen binding fragment thereof is administered at a dose of approximately 9 mg/kg once weekly, and docetaxel is administered at a dose of approximately 75 mg/m² once every three weeks.

In some embodiments, the anti-clusterin antibody or antigen binding fragment thereof is administered at a dose of approximately 9 mg/kg once weekly, and docetaxel is administered at a dose of approximately 60 mg/m² once every three weeks.

In some embodiments, the anti-clusterin antibody or antigen binding fragment thereof is administered at a dose of approximately 6 mg/kg once weekly, and docetaxel is administered at a dose of approximately 75 mg/m² once every three weeks.

In some embodiments, the anti-clusterin antibody or antigen binding fragment thereof is administered at a dose of approximately 6 mg/kg once weekly, and docetaxel is administered at a dose of approximately 60 mg/m² once every three weeks.

In some embodiments, the anti-clusterin antibody or antigen binding fragment thereof is administered at a dose of approximately 3 mg/kg once weekly, and docetaxel is administered at a dose of approximately 75 mg/m² once every three weeks.

In some embodiments, the anti-clusterin antibody or antigen binding fragment thereof is administered at a dose of approximately 3 mg/kg once weekly, and docetaxel is administered at a dose of approximately 60 mg/m² once every three weeks.

In some embodiments, the anti-clusterin antibody or antigen binding fragment thereof is humanized 16B5 and is administered at a dose of 12 mg/kg once weekly, and docetaxel is administered at a dose of 75 mg/m² once every three weeks.

In some embodiments, the anti-clusterin antibody or antigen binding fragment thereof is humanized 16B5 and is administered at a dose of 12 mg/kg once weekly, and docetaxel is administered at a dose of 60 mg/m² once every three weeks.

In some embodiments, the anti-clusterin antibody or antigen binding fragment thereof is humanized 16B5 and is administered at a dose of 9 mg/kg once weekly, and docetaxel is administered at a dose of 75 mg/m² once every three weeks.

In some embodiments, the anti-clusterin antibody or antigen binding fragment thereof is humanized 16B5 and is administered at a dose of 9 mg/kg once weekly, and docetaxel is administered at a dose of 60 mg/m² once every three weeks.

In some embodiments, the anti-clusterin antibody or antigen binding fragment thereof is humanized 16B5 and is administered at a dose of 6 mg/kg once weekly, and docetaxel is administered at a dose of 75 mg/m² once every three weeks.

In some embodiments, the anti-clusterin antibody or antigen binding fragment thereof is humanized 16B5 and is administered at a dose of 6 mg/kg once weekly, and docetaxel is administered at a dose of 60 mg/m² once every three weeks.

In some embodiments, the anti-clusterin antibody or antigen binding fragment thereof is humanized 16B5 and is administered at a dose of 3 mg/kg once weekly, and docetaxel is administered at a dose of 75 mg/m² once every three weeks.

In some embodiments, the anti-clusterin antibody or antigen binding fragment thereof is humanized 16B5 and is administered at a dose of 3 mg/kg once weekly, and docetaxel is administered at a dose of 60 mg/m² once every three weeks.

A cycle of treatment may last, for example, 21 days. During one cycle of treatment, the subject may receive for example, the anti-clusterin antibody or antigen binding fragment thereof once weekly and the docetaxel once every three weeks. The subject may receive two or more consecutive treatment cycles.

In some embodiments, a treatment cycle is considered completed after a period of approximately seven days after a subject has received both the anti-clusterin antibody or antigen binding fragment thereof and docetaxel.

For example, when both the anti-clusterin antibody or antigen binding fragment thereof and docetaxel are administered every week, a treatment cycle is considered to be 7 days.

For example, when the anti-clusterin antibody or antigen binding fragment thereof is administered every week and docetaxel is administered every two weeks, a treatment cycle is considered to be 14 days.

For example, when the anti-clusterin antibody or antigen binding fragment thereof is administered every week and docetaxel is administered every three weeks, a treatment cycle is considered to be 21 days.

In some exemplary embodiments, one treatment cycle is approximately 21 days.

In some exemplary embodiments, essentially all treatment cycles are approximately 21 days.

In some exemplary embodiments, each treatment cycles are approximately 21 days.

In accordance with the present disclosure, the subject may thus receive a new treatment cycle every 21 days.

In accordance with the present disclosure, a subject may receive at least one treatment cycle prior to isolation of TILs.

In accordance with the present disclosure, a subject may receive at least two treatment cycles prior to isolation of TILs.

In accordance with the present disclosure, a subject may receive at least three treatment cycles prior to isolation of TILs.

In accordance with the present disclosure, a subject may receive at least four treatment cycles prior to isolation of TILs.

In accordance with the present disclosure, a subject may receive four or more treatment cycles prior to isolation of TILs.

In accordance with the present disclosure, a subject may receive at least five treatment cycles prior to isolation of TILs.

In accordance with the present disclosure, a subject may receive at least six treatment cycles prior to isolation of TILs.

In accordance with the present disclosure, a subject may receive at least seven treatment cycles prior to isolation of TILs.

In accordance with the present disclosure, a subject may receive at least eight treatment cycles prior to isolation of TILs.

In accordance with the present disclosure, a subject may receive at least nine treatment cycles prior to isolation of TILs.

In accordance with the present disclosure, a subject may receive at least ten treatment cycles prior to isolation of TILs.

In accordance with the present disclosure, a subject may receive at least eleven treatment cycles prior to isolation of TILs.

In accordance with the present disclosure, a subject may receive at least twelve treatment cycles prior to isolation of TILs.

In accordance with the present disclosure, a subject may receive at least thirteen treatment cycles prior to isolation of TILs.

In accordance with the present disclosure, a subject may receive at least fourteen treatment cycles prior to isolation of TILs.

In accordance with the present disclosure, a subject may receive at least fifteen treatment cycles prior to isolation of TILs.

In accordance with the present disclosure, a subject may receive at least sixteen treatment cycles prior to isolation of TILs.

In accordance with the present disclosure, a subject may receive at least seventeen treatment cycles prior to isolation of TILs.

In accordance with the present disclosure, a subject may receive at least eighteen treatment cycles prior to isolation of TILs.

In accordance with the present disclosure, a subject may receive at least nineteen treatment cycles prior to isolation of TILs.

In accordance with the present disclosure, a subject may receive at least twenty treatment cycles prior to isolation of TILs.

In accordance with the present disclosure, a subject may receive more than twenty treatment cycles prior to isolation of TILs.

In accordance with the present disclosure, a subject may receive at least one treatment cycle after infusion of TILs.

In accordance with the present disclosure, a subject may receive at least two treatment cycles after infusion of TILs.

In accordance with the present disclosure, a subject may receive at least three treatment cycles after infusion of TILs.

In accordance with the present disclosure, a subject may receive at least four treatment cycles after infusion of TILs.

In accordance with the present disclosure, a subject may receive four or more treatment cycles after infusion of TILs.

In accordance with the present disclosure, a subject may receive at least five treatment cycles after infusion of TILs.

In accordance with the present disclosure, a subject may receive at least six treatment cycles after infusion of TILs.

In accordance with the present disclosure, a subject may receive at least seven treatment cycles after infusion of TILs.

In accordance with the present disclosure, a subject may receive at least eight treatment cycles after infusion of TILs.

In accordance with the present disclosure, a subject may receive at least nine treatment cycles after infusion of TILs.

In accordance with the present disclosure, a subject may receive at least ten treatment cycles after infusion of TILs.

In accordance with the present disclosure, a subject may receive at least eleven treatment cycles after infusion of TILs.

In accordance with the present disclosure, a subject may receive at least twelve treatment cycles after infusion of TILs.

In accordance with the present disclosure, a subject may receive at least thirteen treatment cycles after infusion of TILs.

In accordance with the present disclosure, a subject may receive at least fourteen treatment cycles after infusion of TILs.

In accordance with the present disclosure, a subject may receive at least fifteen treatment cycles after infusion of TILs.

In accordance with the present disclosure, a subject may receive at least sixteen treatment cycles after infusion of TILs.

In accordance with the present disclosure, a subject may receive at least seventeen treatment cycles after infusion of TILs.

In accordance with the present disclosure, a subject may receive at least eighteen treatment cycles after infusion of TILs.

In accordance with the present disclosure, a subject may receive at least nineteen treatment cycles after infusion of TILs.

In accordance with the present disclosure, a subject may receive at least twenty treatment cycles after infusion of TILs.

In accordance with the present disclosure, a subject may receive more than twenty treatment cycles after infusion of TILs.

In some embodiments, the anti-clusterin antibody or antigen binding fragment thereof is administered by infusion over approximately a 1-hour time frame.

In some embodiments, docetaxel is administered by infusion over approximately a 1-hour time frame.

In accordance with the present disclosure, the anti-clusterin antibody or antigen binding fragment thereof and docetaxel are administered on same day.

The anti-clusterin antibody or antigen binding fragment thereof and docetaxel may be administered separately.

The anti-clusterin antibody or antigen binding fragment thereof and docetaxel may be administered sequentially.

In some embodiments, the anti-clusterin antibody or antigen binding fragment thereof is administered by infusion over approximately a 1-hour time frame and docetaxel is subsequently administered by infusion on same day over approximately a 1-hour time frame.

In some embodiments, docetaxel is administered by infusion over approximately a 1-hour time frame and the anti-clusterin antibody or antigen binding fragment thereof is subsequently administered by infusion on same day over approximately a 1-hour time frame.

### Example 1—Effect of AB-16B5 on Infiltration of Immune Cells in the Tumor Microenvironment

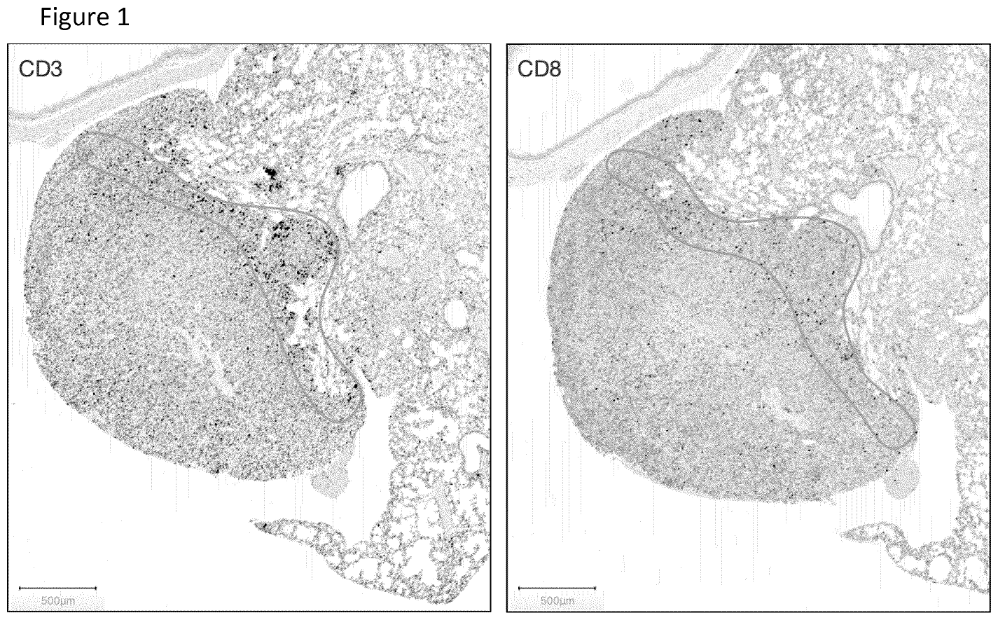

Balb/c mice were orthotopically implanted with 5×10⁵ 4T1 cells in the 4^{th} mammary fat pad. Animals received IP saline treatment thrice weekly. The primary tumor was surgically removed at Day 16 post-implantation. The animals were sacrificed at Day 36 and the lungs were excised. Tissues were fixed in paraformaldehyde and processed for paraffin embedding. Tissue sections were probed with anti-mouse CD3, anti-mouse CD8 and anti-mouse B220 antibodies. Signals were revealed with specific secondary antibodies conjugated with horseradish peroxidase and counter stained with hematoxylin and eosin. Results presented in FIG. 1 indicate that the 4T1 lung metastases create an immune cold microenvironment which prevents the infiltration of B and T lymphocytes in tumors. Delineated regions indicate that CD3 and CD8 T lymphocytes are restricted in the tumor margin as a consequence of EMT.

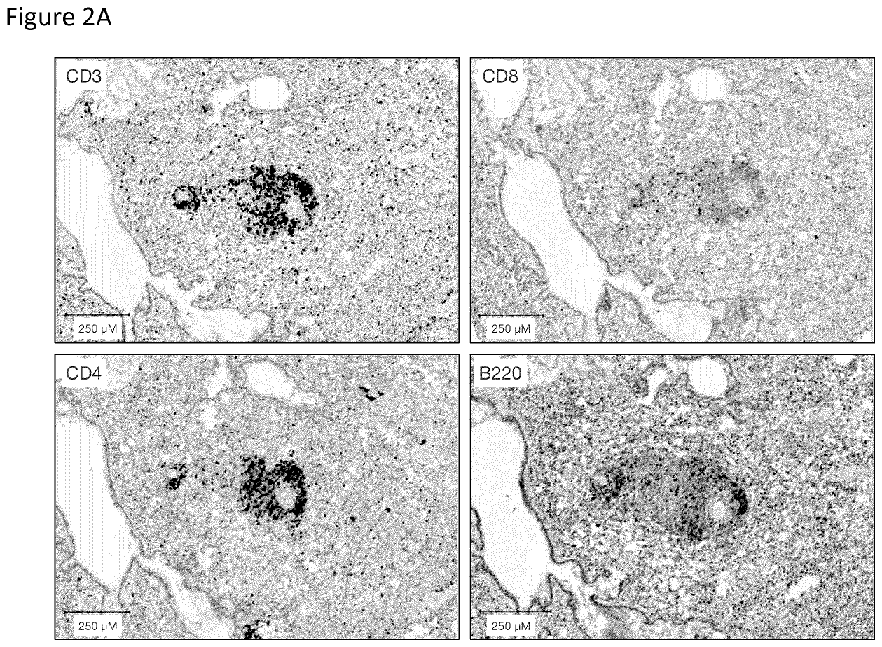

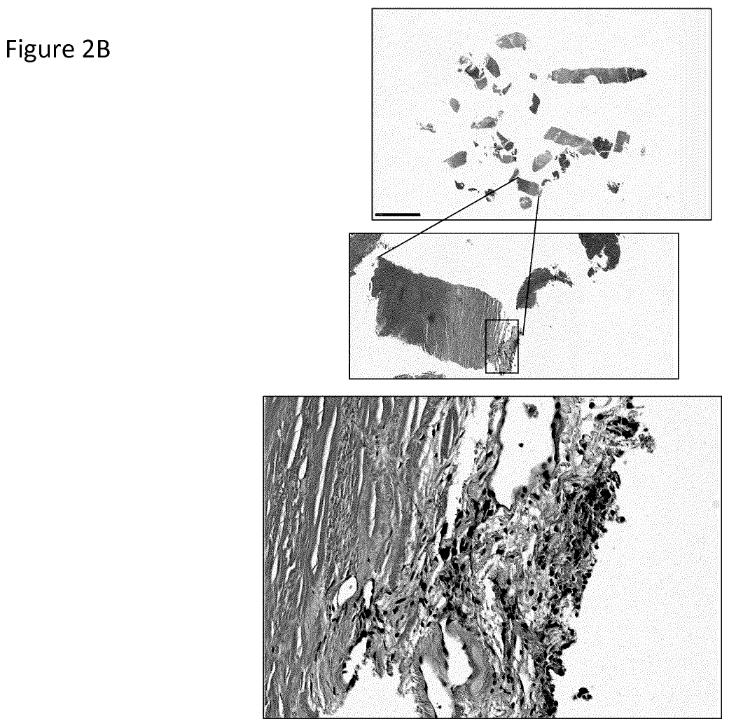

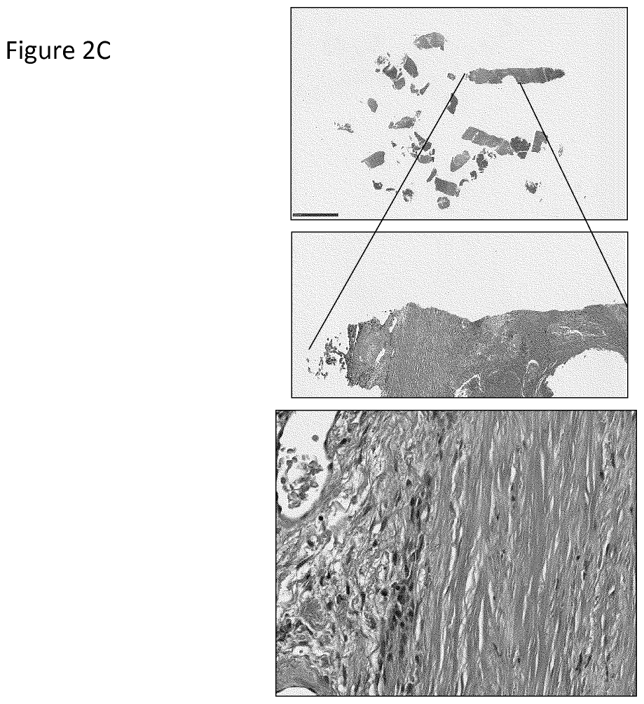

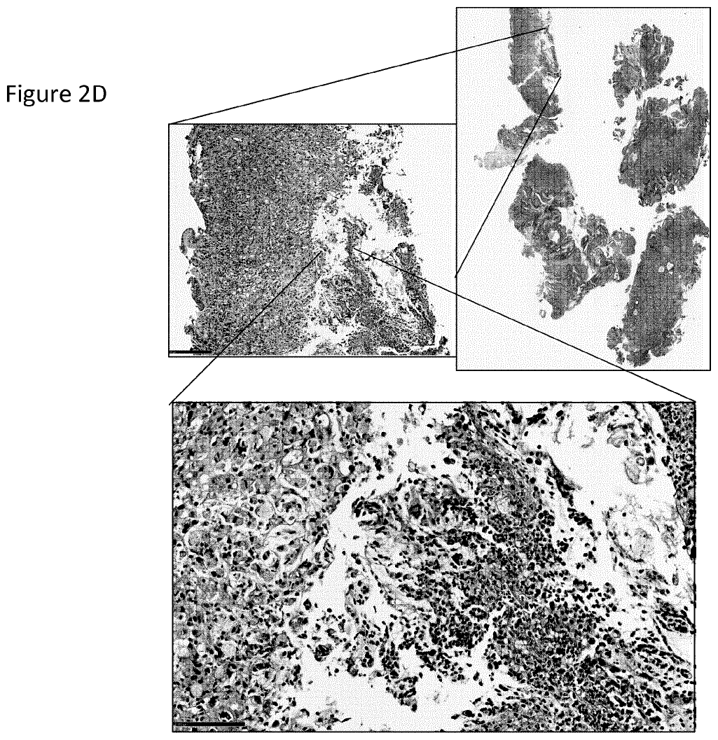

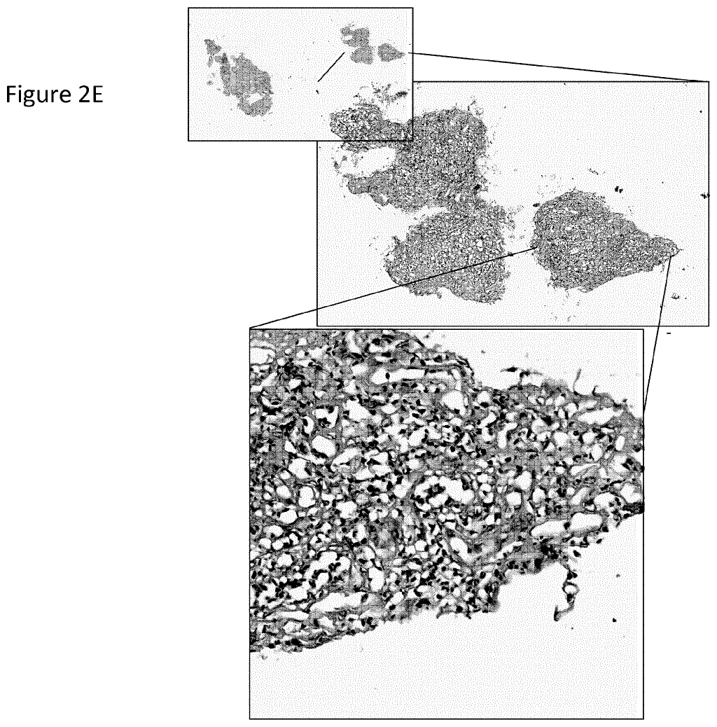

Animals bearing 4T1 tumors were treated with the AB-16B5 antibody (murine 16B5) thrice a week IP at 10 mg/kg. The primary tumor was surgically removed at Day 16 post-implantation. The animals were sacrificed at Day 36 and the lungs were excised. Tissues were fixed in paraformaldehyde and processed for paraffin embedding. Tissue sections were probed with anti-mouse CD3, anti-mouse CD8 and anti-mouse B220 antibodies. Signals were revealed with specific secondary antibodies conjugated with horseradish peroxidase and counter stained with hematoxylin and eosin. Results presented in FIG. 2 indicate that there were fewer and much smaller lung metastases that were densely infiltrated with CD3 and CD8 T cells. There was also evidence of plasmocytes penetration in 16B5-treated tumors.

AB-16B5 thus allows infiltration of immune cells in the tumor microenvironment in immunocompetent mice. AB-16B5 might represent a new therapeutic avenue to create a warmer tumor environment to stimulate a strong immune response against tumors.

In parallel human tumor biopsies of patients treated with AB-16B5 (humanized 16B5) as single agent were analyzed (FIGS. 2B-2E). Needle biopsies obtained from a patient with metastatic thyroid cancer and form a patient with inoperable metastatic gastric cancer were sectioned and stained with hematoxylin and eosin. An on-treatment biopsy from a patient with thyroid cancer metastasic to the lungs was obtained after the second cycle treatment with AB-16B5. As shown in FIG. 2B, essentially all tumor fragments were necrotic. A lymphoplasmacytic infiltrate was observed along the edge of the fragment on display. Hemosiderin-laden macrophages were observed inside necrotic areas some reflecting red blood cell extravasation associated with necrosis (not shown). FIG. 2C shows a perivascular infiltrate composed of plasma cells along the edge of tumor fragment from the same patient. The analysis of the pre-treatment biopsy from the metastatic gastric cancer case showed several fragments of gastric mucosa infiltrated by a diffuse poorly differentiated gastric cancer (signet-ring cells) The fragment on display showed foci of necrosis with a predominantly acute neutrophilic infiltrate. FIG. 2E shows the on-treatment biopsy obtained after the second cycle of treatment with AB-16B5 comprised of three tumor fragments. The larger fragment consisted of normal superficial gastric mucosa and that the small fragments were infiltrated by a mix neutrophilic and mononucleated immune cells infiltrate.

### Example 2—Effect of the Combination Therapy of AB-16B5 and Docetaxel on Infiltration of Immune Cells in the Tumor Microenvironment

An immunocompetent mouse cancer model was selected for testing the extent of the immune response upon treatment with AB-16B5 monotherapy or combination of AB-16B5 and docetaxel using the murine 16B5.

Five groups, each consisting of 10 female Balb/c mice were assigned to this study (see Table 1 below). All animals received subcutaneous implantation of 4T1 mouse mammary carcinoma cells in the 4th inguinal mammary gland. Treatment was initiated on the day of implantation (defined as Day 1). Animals from Group 1 (Gr. 1) received IP treatment of saline vehicle control twice a week for the duration of the study. Animals from Group 2 (Gr. 2) received 10 mg/kg of docetaxel weekly for five weeks by IP administration. Animals from Group 3 (Gr. 3) received 10 mg/kg of docetaxel weekly for two weeks and 10 mg/kg of AB-16B5, twice weekly for five weeks. Animals from Group 4 (Gr. 4) received 10 mg/kg of docetaxel weekly and 5 mg/kg of 16B5 twice weekly each over the course of a five weeks treatment. Animals from Group 5 (Gr. 5) received AB-16B5 twice weekly for five weeks. On Day 36, the primary tumors were excised and on Day 37, the animals were sacrificed and the number of grossly visible metastatic nodules on the surface of the lungs was counted.

TABLE 1
Dosing schedule:

| | | | | | Week 3 to 5 | | |
|---|---|---|---|---|---|---|---|
| | | Week 1 and 2 | | | | Day 2 | |
| Group # | Treatment | Day 1 | Day 2 | Day 4 | Day 1 | or 3* | Day 4 |
| Group 1 | Vehicle IP, 2X/week | Vehicle | | Vehicle | Vehicle | | Vehicle |
| | for 5 weeks | | | | | | |
| Group 2 | Docetaxel 10 mg/kg | | DTX | | | DTX | |
| | (IP, 1X/week for 5 weeks | | | | | | |
| Group 3 | Docetaxel 10 mg/kg | AB16B5 | DTX | AB16B5 | AB16B5 | | AB16B5 |
| | (IP, 1X/week for 2 weeks) | | | | | | |
| | and AB-16B5 10 mg/kg | | | | | | |
| | (IP, 2X/week for 5 weeks) | | | | | | |
| Group 4 | Docetaxel 10 mg/kg | AB16B5 | DTX | AB16B5 | AB16B5 | DTX | AB16B5 |
| | (IP, 1X/week for 5 weeks) | | | | | | |
| | and AB-16B5 10 mg/kg | | | | | | |
| | (IP, 2X/week for 5 weeks) | | | | | | |
| Group 5 | AB-16B5 10 mg/kg | AB16B5 | | AB16B5 | AB16B5 | | AB16B5 |
| | (IP, 2X/week for 5 weeks) | | | | | | |

*2 animals from Group 4 received docetaxel on the same day as AB16B5 (day 4) on week 3

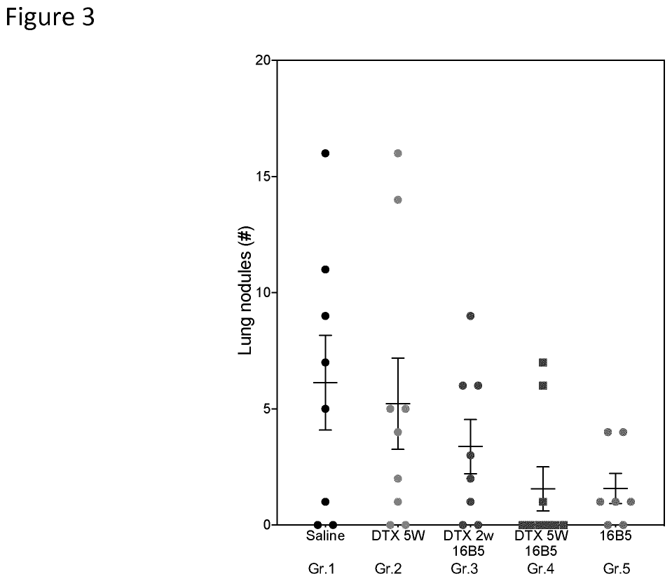

Results presented in FIG. 3, show that lungs of the animals from Group 4 and Group 5 comprised less metastatic lung nodules than the saline-control-treated mice. As well, mice that were treated in monotherapy with docetaxel had as many metastatic lung nodules as the saline control group. Treatment with docetaxel for two weeks in combination with 16B5 led to fewer metastatic lung nodules that in Group 1 and Group 2 but the response to treatment was not as extensive as in Group 4 and Group 5. There were more animals in Group 4 for which no nodules could be detected than in any other groups. These results suggest that AB-16B5 monotherapy or combination therapy with docetaxel effectively inhibit metastatic invasion in immunocompetent mice. These results also suggest that it may be preferable to administer AB-16B5 and docetaxel over the entire course of treatment.

The primary tumors excised at Day 16 post implantation, were processed with collagenase and hyaluronidase and immune cells were purified by positive selection using magnetic latex beads coated with an anti-CD45 antibody. The purified cells were transferred into small petri dishes containing culture medium supplemented with IL2 and IL7 to perform phenotypic analyses. It was found that very few CD45+ were present in the primary tumors retrieved from Group 1 and Group 2 animals. In contrast, there were more immune cells in tumors retrieved from Group 3, Group 4 and Group 5 animals.

Treatment of mice implanted with 4T1 tumor cells with docetaxel (DTX 5W) was relatively ineffective. The 4T1 tumors bear an EMT-high signature that causes resistance to many chemotherapeutic agents including docetaxel. Treatment of mice with docetaxel for 2 weeks and with 16B5 for 5 weeks was not as effective as treatment with 16B5 in monotherapy possibly because transient exposure of tumors to docetaxel resulted in increased resistance of tumors. The combination of docetaxel with 16B5 for 5 weeks proved to be the most effective therapeutic regimen. The combined increase of shed antigens caused by docetaxel and inhibition of EMT resulted in an increased immune response that translated in fewer lung metastases in this group compared to 16B5 in monotherapy.

AB-16B5 in monotherapy and the combination of AB-16B5 with docetaxel thus allow infiltration of immune cells in the tumor microenvironment in immunocompetent mice.

### Example 3—Characterization, Purification and Production of Tumor Infiltrating Lymphocytes

Balb/c mice were orthotopically implanted with 5×10⁵ 4T1 cells in the 4th mammary fat pad. Animals received intraperitoneal (IP) AB-16B5 (murine 16B5) 10 mg/kg twice weekly in combination with IP docetaxel 10 mg/kg weekly (Group 15: animals 1501, 1502 and 1503) or IP AB-16B5 10 mg/kg twice weekly (Group 25: animal). The primary tumor was surgically removed at Day 16 post-implantation. The animals were sacrificed at Day 36 and the lungs were excised and each visible lung metastasis was carefully dissected. Each visible metastatic nodule, if any, was excised and processed for a rapid expansion of tumor infiltrating lymphocyte protocol. The metastatic nodules were sectioned into small pieces of 2-3 mm edge that were individually grown in 24 well plates containing culture medium supplemented with FBS, IL2, IL7, ITS (1,000 U/mL IL2, 2.0 ng/ml IL7 and 1× insulin—transferrin—selenium cocktail (Gibco 41400-045)).

After three weeks in culture, 100,000 cells were taken from each of the lymphocyte cultures (six cultures corresponding to three animals from Group 15 and three animals from Group 25) and directly placed in culture with 100,000 4T1 tumor cells. After overnight co-culture, the supernatant was recovered for INFγ quantification by ELISA.

The results of INFγ secretion from lymphocyte cultures in the presence of 4T1 cells indicate that lymphocytes isolated from lung metastatic nodules secrete INFγ at high levels with highest average levels observed in the docetaxel-16B5 group (see Table 2). These results confirm that inhibition of EMT with the anti-sCLU 16B5 mAb contributes to the generation of a “warm” tumor microenvironment that allows the infiltration of T lymphocytes in tumors.

TABLE 2

| Sample | INFγ pg/mL |
|---|---|
| 1501 | 5370.0 |
| 1502 | 12488.8 |
| 1503 | 2326.3 |
| 2501 | 8538.8 |
| 2502 | 3770.0 |
| 2503 | 4538.8 |

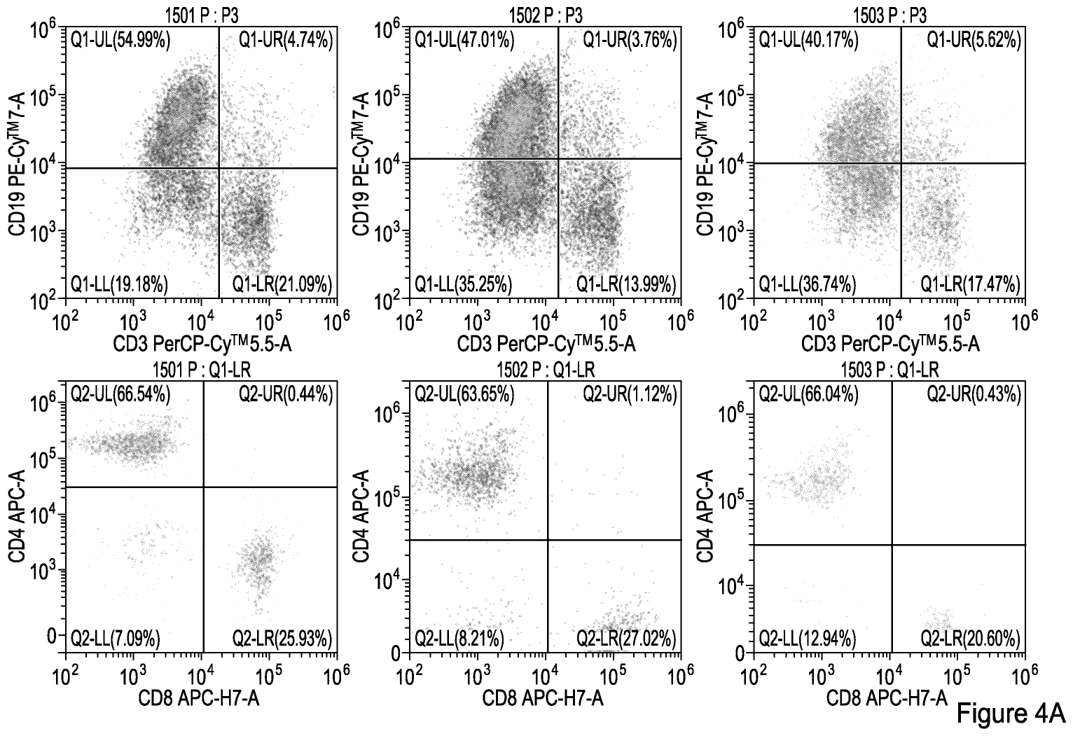

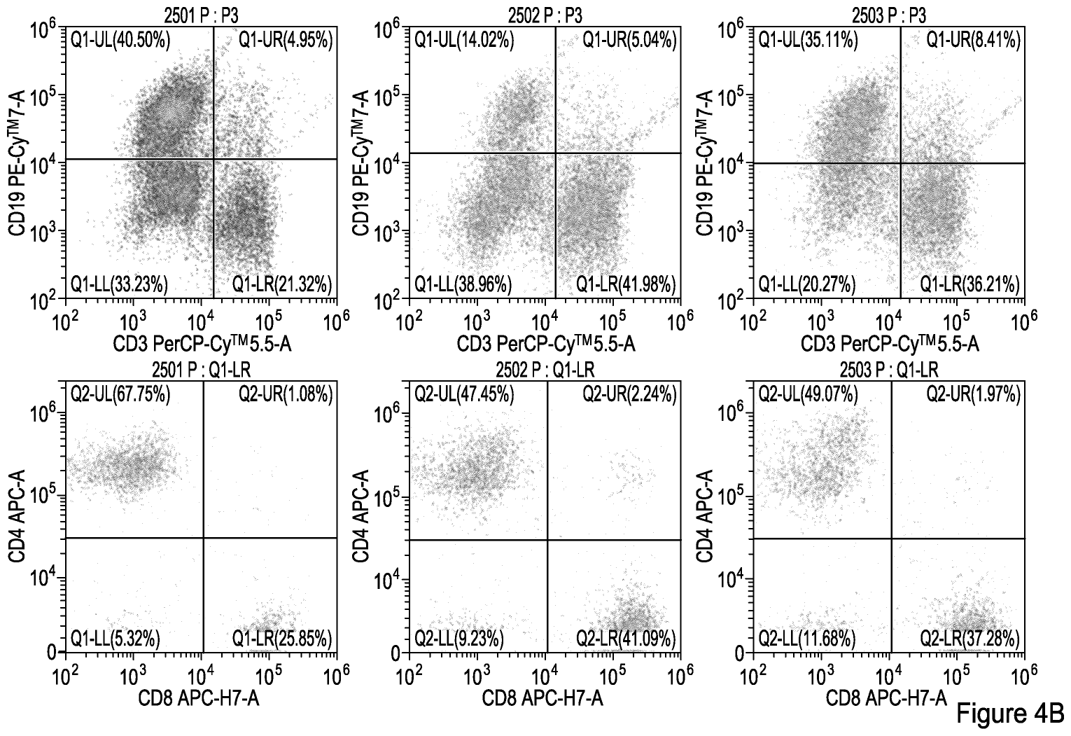

The lymphocytes were stimulated with anti-CD3 and anti-CD28 monoclonal antibodies. Lymphocytes from each donor animal were pooled and processed for flow cytometry analysis with antibodies against CD45 (lymphocyte common antigen), CD3, CD4, CD8 and CD19 (B-cell biomarker) (FIG. 4A and FIG. 4B). The resulting single cell preparations were initially selected on their size to select those corresponding to immune cells. They were further gated on an FSC/SSC plot to exclude dead cells and debris. Flow cytometric analyses were then performed with antibodies against CD45, CD3, CD19, CD3, CD4 and CD8. Immune cells positive for CD45 were gated on CD3 and CD19 (P3). The CD3+ cells were further gated on CD4 and CD8 (Q1-LR).

Results indicated 80-90% cell viability of CD45+ cells for both groups. The CD45+ cells from group 15 (FIG. 4A) comprised 40.2% to 55.0% of CD19 cells and 14.0% to 21.1% of CD3+ cells. The CD3+ cells comprised 63.7% to 66.5% of CD4+ T cells and 20.6% to 27.0% CD8+ T cells. The CD45+ cells from group 25 (FIG. 4B) comprised 14.0% to 35.0% of CD19 cells and 21.3% to 42.0% of CD3+ cells. The CD3+ cells comprised 47.5% to 67.8% of CD4+ T cells and 25.9% to 41.1% CD8+ T cells.

### Example 4—Evaluation of the Immunoreactivity of Tumor Infiltrating T Lymphocytes from Mice Treated with AB-16B5 in Combination with Docetaxel

Balb/c mice were orthotopically implanted with 5×10⁵ 4T1 cells in the 4th mammary fat pad. Animals received intraperitoneal (IP) AB-16B5 (murine 16B5) 10 mg/kg twice weekly in combination with IP docetaxel 10 mg/kg weekly. The primary tumor was surgically removed at Day 21 post-implantation. The animals were sacrificed at Day 36 and the lungs were excised and each visible lung metastasis was carefully dissected. 18 lymphocyte cultures containing 1 to 3 small lung metastases in a 24-well G-Rex multi well plate (Wilson-Wolf #80192M). TILS were expanded in defined R&D Systems™ ExCellerate Human T Cell Expansion Media (#CCM030) containing 600 IU/mL IL2. After three weeks of culture, 100,000 cells were taken from each TILs culture, washed in PBS and placed in culture with 100,000 4T1 tumor cells. After overnight co-culture, the supernatant was recovered and the concentration of INFγ was evaluated by ELISA. The results of INFγ secretion from TILs cultures in the presence of 4T1 cells indicate that lymphocytes isolated from lung metastatic nodules produce INFγ at varying levels. Based on the current literature, it was established that T cells cultures in which the levels of IFNγ were lower than 300 pg/mL were considered weak: those between and including 300 pg/mL to 500 pg/mL were considered intermediate and those above 500 pg/mL were considered high (see Table 3).

TABLE 3

| Sample | INFγ pg/mL |
|---|---|
| 1 | 1316 |
| 2 | 1901 |
| 3 | 226 |
| 4 | 110 |
| 5 | 192 |
| 6 | 1022 |
| 7 | 590 |
| 8 | 2761 |
| 9 | 461 |
| 10 | 1145 |
| 11 | 436 |
| 12 | 523 |
| 13 | 799 |
| 14 | 775 |
| 15 | 424 |
| 16 | 687 |
| 17 | 913 |
| 18 | 297 |

As evidenced from Table 3, all TILs cultures had INFγ secretion levels of equal to or higher than 100 pg/ml. Fourteen out of these TILs cultures showed INFγ secretion levels of equal to or higher than 300 pg/ml and eleven out of eighteen TILs cultures showed INFγ secretion levels of equal to or higher than 500 pg/ml.

The lymphocytes were further analyzed by flow cytometry. They were stimulated with anti-CD3 and anti-CD28 monoclonal antibodies. Lymphocytes from each culture were processed for flow cytometry analysis with antibodies against CD45, CD3, CD4 and CD8. The resulting single cell preparations were initially selected on their size to select those corresponding to immune cells. They were further gated on an FSC/SSC plot to exclude dead cells and debris. Flow cytometric analyses were then performed with antibodies against CD45, CD3, CD4 and CD8. Immune cells positive for CD45 were gated on CD3. Results indicated that 73% to 95% of the viable cells were CD3 positive. The CD3+ cells were further gated on CD4 and CD8. Results indicated that the conditions used to grow immunoreactive TILs were favorable to enrich for CD8+ T cells. Interestingly, cultures with low IFNγ production such as #3 and #5 had a higher content of CD4+ T cells that could suggest the presence of CD4+ regulatory T cells.

TABLE 4

| | | | Uncharacterized |
|---|---|---|---|
| Sample | CD8+ | CD4+ | CD4− CD8− |
| 1 | 94% | 1% | 5% |
| 2 | 69% | 1% | 29% |
| 3 | 61% | 18% | 21% |
| 4 | 79% | 2% | 19% |
| 5 | 39% | 12% | 49% |
| 6 | 69% | 4% | 27% |
| 7 | 93% | 2% | 5% |
| 8 | 77% | 3% | 20% |
| 9 | 73% | 0% | 27% |
| 10 | 74% | 1% | 25% |
| 11 | 76% | 0% | 24% |
| 12 | 73% | 1% | 26% |
| 13 | 73% | 0% | 27% |
| 14 | 54% | 0% | 46% |
| 15 | 56% | 1% | 43% |
| 16 | 61% | 1% | 38% |
| 17 | 63% | 1% | 36% |
| 18 | 47% | 1% | 52% |

### Example 5—Detailed Rapid Expansion Protocol of TILs

The following rapid expansion protocol is derived from Jin J., et al., J Immunother. 35(3): 283-292, 2012 (the entire content of which is incorporated herein by reference).

### Initial Culture Phase

TILs are initially cultured from enzymatic tumor digests and tumor fragments (1-8 mm³) produced by sharp dissection. Tumor digests are generated by incubation in enzyme media (RPMI 1640, 2 mM Glutmax, 10 ug/mL gentamicin, 30 units/mL DNase and 1.0 mg/mL collagenase) followed by mechanical dissociation (GentleMACS, Miltenyi Biotec, Auburn, CA). Immediately after placing the tumor in enzyme media, it is mechanically dissociated for approximately 1 minute. The solution is then incubated for 30 minutes at 37° C. in 5% CO₂ and then mechanically disrupted again for approximately 1 minute. After being incubated again for 30 minutes at 37° C. in 5% CO₂, the tumor is mechanically disrupted a third time for approximately one minute. If after the third mechanical disruption, large pieces of tissue are present, one or two additional mechanical dissociations are applied to the sample, with or without 30 additional minutes of incubation at 37° C. in 5% CO₂. At the end of the final incubation if the cell suspension contained a large number of red blood cells or dead cells, a density gradient separation using ficoll is performed to remove these cells.

When TIL cultures are initiated in 24-well plates (Costar 24 well cell culture cluster, flat bottom, Corning Incorporated, Corning, NY), each well are seeded with 1×10⁶ tumor digest cells or one tumor fragment approximately 1 to 8 mm³ in size in 2 mL of complete medium (CM) with IL-2 (6000 IU/mL, Chiron Corp., Emeryville, CA). CM consisted of RPMI 1640 with glutamine, supplemented with 10% human AB serum, 25 mM Hepes and 10 μg/mL gentamicin. When cultures are initiated in gas-permeable flasks with a 40 mL capacity and a 10 cm² gas-permeable silicon bottom (G-Rex10, Wilson Wolf Manufacturing, New Brighton, MN, USA) (FIG. 1), each flask is loaded with 10 to 40×10⁶ viable tumor digest cells or 5 to 30 tumor fragments in 10 to 40 mL of CM with IL-2 (recombinant human IL-2). Both the G-Rex10 and 24-well plates are incubated in a humidified incubator at 37° C. in 5% CO₂ and five days after culture initiation, half the media is removed and replaced with fresh CM and IL-2 and after day 5, half the media is changed every 2 to 3 days.

### Expansion Phase

Rapid expansion protocol (REP) of TILs is performed using T-175 flasks and gas permeable bags or gas permeable G-Rex®) flasks. For TIL REP in T-175 flasks, 1×10⁶ TIL suspended in 150 mL of media is added to each T-175 flask. The TILs are cultured with irradiated (50 Gy) allogeneic peripheral blood mononuclear cells (PBMC) as “feeder” cells at a ratio of 1 to 100 and the cells are cultured in a 1 to 1 mixture of CM and AIM-V medium (50/50 medium), supplemented with 3000 IU per mL of IL-2 and 30 ng per mL of anti-CD3. The T-175 flasks are incubated at 37° C. in 5% CO₂. Half the media is changed on day 5 using 50/50 medium with 3000 IU per mL of IL-2. On day 7 cells from two T-175 flasks are combined in a 3 liters bag and 300 mL of AIM V with 5% human AB serum and 3000 IU per mL of IL-2 are added to the 300 mL of TIL suspension. The number of cells in each bag is counted every day or two and fresh media is added to keep the cell count between 0.5 and 2.0×10⁶ cells/mL.

For TIL REP in 500 ml capacity flasks with 100 cm² gas-permeable silicon bottoms (G-Rex100, Wilson Wolf) (FIG. 1) 5×10⁶ or 10×10⁶ TILs are cultured with irradiated allogeneic PBMC at a ratio of 1 to 100 in 400 mL of 50/50 medium, supplemented with 5% human AB serum, 3000 IU per mL of IL-2 and 30 ng per ml of anti-CD3. The G-Rex100 flasks are incubated at 37° C. in 5% CO₂. On day 5, 250 mL of supernatant is removed and placed into centrifuge bottles and centrifuged at 1500 rpm (491×g) for 10 minutes. The TIL pellets are re-suspended with 150 mL of fresh medium with 5% human AB serum, 3000 IU per mL of IL-2, and added back to the original G-Rex100 flasks. When TIL are expanded serially in G-Rex100 flasks, on day 7 the TIL in each G-Rex100 are suspended in the 300 mL of media present in each flask and the cell suspension is divided into 3 100 mL aliquots that are used to seed 3 G-Rex100 flasks. Then 150 mL of AIM-V with 5% human AB serum and 3000 IU per mL of IL-2 is added to each flask. The G-Rex100 flasks are incubated at 37° C. in 5% CO₂ and after 4 days 150 mL of AIM-V with 3000 IU per mL of IL-2 is added to each G-Rex100 flask. The cells are harvested on day 14 of culture.

Wardell et al., (US publication No. 2018/0282694, the entire content of which is incorporated herein by reference) disclose an improved and shortened process for expanding TILs and producing a therapeutic population of TILs which is applicable to the present disclosure.

If desired an anti-CD28 antibody and/or an anti-4-1B may be added during the expansion phase.

### Cell Counts, Viability, Flow Cytometry

The expression of CD3, CD4, CD8 and CD56 is measured by flow cytometry with antibodies from BD Biosciences (BD Biosciences, San Jose, CA) using a FACSCanto flow cytometer (BD Biosciences). The cells are counted manually using a disposable c-chip hemacytometer (VWR, Batavia, IL) and viability is assessed using trypan blue staining.

### Cytokine Release Assays

TILs are evaluated for interferon-gamma (IFN-γ) secretion in response to stimulation either with OKT3 antibody or co-culture with autologous tumor digest. For OKT3 stimulation, TILs are washed extensively, and duplicate wells are prepared with 1× 10⁵ cells in 0.2 ml CM in 96 well flat-bottom plates pre-coated with 0.1 or 1.0 μg/mL of OKT-3 antibody diluted in PBS. After overnight incubation, the supernatants are harvested and IFN-γ in the supernatant is measured by ELISA (Pierce/Endogen, Woburn, MA). For the co-culture assay, TIL cells are placed into a 96-well plate with autologous tumor cells. After a 24 hour incubation, supernatants are harvested and IFN-γ release was quantified by ELISA.

The embodiments and examples described herein are illustrative and are not meant to limit the scope of the claims. Variations of the foregoing embodiments, including alternatives, modifications and equivalents, are intended by the inventors to be encompassed by the claims. Citations listed in the present application are incorporated herein by reference.

### REFERENCES

Al-Lazikani et al., Standard conformations for the canonical structures of immunoglobulins. J Mol Biol 273:927-948, 1997.
Brochet et al. IMGT/V-QUEST: the highly customized and integrated system for IG and TR standardized V-J and V-D-J sequence analysis. Nucl Acids Res 36:W503-W508, 2008.
Andrew C. R. Martin, Antibody Engineering Vol. 2, Chapter 3: Protein Sequence and Structure Analysis of Antibody Variable Domains. R. Kontermann and S. Dubel (eds.), DOI 10.1007/978-3-642-01147-4_3, #Springer-Verlag Berlin Heidelberg 2010
Shibue, T., Weinberg, R. EMT, CSCs, and drug resistance: the mechanistic link and clinical implications. Nat Rev Clin Oncol 14: 611-629 (2017).
Terry, S., Savagner, P., Ortiz-Cuaran, S., Mahjoubi, L., Saintigny, P., Thiery, J.-P. and Chouaib, S., New insights into the role of EMT in tumor immune escape. Mol Oncol, 11: 824-846 (2017).
Lenferink, A., Cantin, C., Nantel, A. et al. Transcriptome profiling of a TGF-ß-induced epithelial-to-mesenchymal transition reveals extracellular clusterin as a target for therapeutic antibodies. Oncogene 29: 831-844 (2010).
New response evaluation criteria in solid tumours: Revised RECIST guideline (version 1.1)” E. A. Eisenhauer, P. Therasse, J. Bogaerts, L. H. Schwartz, D. Sargent, R. Ford, J. Dancey, S. Arbuck, S. Gwyther, M. Mooney, L. Rubinstein, L. Shankar, L. Dodd, R. Kaplan, D. Lacombe, J. Verweij: Eur J Cancer, 45 (2009) 228-24.
Cristiano Ferrario, Julie Laurin, Leon Van Kempen, Caroline Lambert, Alan Spatz, Oksana Markova, Gerald Batist, Adrian Langleben, Mario Filion, Jacques Jolivet. Phase 1 first-in-human study of anti-clusterin antibody AB-16B5 in patients with advanced solid malignancies [abstract]. In: Proceedings of the American Association for Cancer Research Annual Meeting 2017: 2017 Apr. 1-5: Washington, DC. Philadelphia (PA): AACR: Cancer Res 2017:77(13 Suppl): Abstract nr CT098. doi: 10.1158/1538-7445.AM2017-CT098.
Hodge, J. W. Garnett, C. T., Farsaci, B., et al. Chemotherapy-induced immunogenic modulation of tumor cells enhances killing by cytotoxic T lymphocytes and is distinct from immunogenic cell death. Int. J. Cancer. 133: 624-636 (2013).
Jiang X, Dudzinski S, Beckermann K E, et al. MRI of tumor T cell infiltration in response to checkpoint inhibitor therapy. Journal for ImmunoTherapy of Cancer 2020:8:e000328. doi: 10.1136/jitc-2019-000328.
MacCallum, R. M., Martin, A. C. R. and Thornton, J. T. ‘Antibody-antigen interactions: Contact analysis and binding site topography’ J. Mol. Biol. 262:732-745, 1996.
Wu and Kabat, An analysis of the sequences of the variable regions of Bence Jones proteins and myeloma light chains and their implications for antibody complementarity. J Exp Med 132:211-250, 1993.

TABLE 5
SEQUENCE LISTING TABLE

| | | SEQ ID |
|---|---|---|
| ID | SEQUENCE | NO: |
| 16B5 CDRL1 | KSSQSLLNSRTRKNYLA | 1 |
| 16B5 CDRL2 | WASTRES | 2 |
| 16B5 CDRL3 | KQSYNLWT | 3 |
| 16B5 CDRH1_v1 | GFNIKDIYMH | 4 |
| 16B5 CDRH2_v1 | RIDPAYGNTKYDPKFQG | 5 |
| 16B5 CDRH3_v1 | RYDTAMDY | 6 |
| 16B5 CDRH1_v2 | GFNIKDIY | 35 |
| 16B5 CDRH2_v2 | IDPAYGNT | 36 |
| 16B5 CDRH3_v2 | ARRYDTAMDY | 37 |
| murine 16B5 VL | DIVMSQSPSSLAVSAGEKVTMSCKSSQSLLNSRTRKN | 7 |
| | YLAWYQQKPGQSPKLLIYWASTRESGVPDRFTGSGSG | |
| | TDFTLTISSVQAEDLAVYYCKQSYNLWTFGGGTKLEF | |
| | K | |
| murine 16B5 VH | EVQLQQSGAELVKPGASVRLSCTTSGFNIKDIYMHWV | 8 |
| | KQRPEQGLEWIGRIDPAYGNTKYDPKFQGKATITADT | |
| | SSNTAYLQLSSLTSEDTAVYYCARRYDTAMDYWGQG | |
| | TSVTVSS | |
| Humanized 16B5 | DIVMTQSPDSLAVSLGERATINCKSSQSLLNSRTRKNY | 9 |
| VL | LAWYQQKPGQPPKLLIYWASTRESGVPDRFSGSGSGT | |
| | DFTLTISSLQAEDVAVYYCKQSYNLWTFGQGTKLEIK | |
| Humanized 16B5 | QVQLVQSGAEVKKPGATVKISCKVSGFNIKDIYMHW | 10 |
| VH | VQQAPGKGLEWMGRIDPAYGNTKYDPKFQGRVTITA | |
| | DTSTDTAYMELSSLRSEDTAVYYCARRYDTAMDYWG | |
| | QGTLVTVSS | |
| Humanized 16B5 | DIVMTQSPDSLAVSLGERATINCKSSQSLLNSRTRKNYL | 11 |
| light chain | AWYQQKPGQPPKLLIYWASTRESGVPDRFSGSGSGTDF | |
| | TLTISSLQAEDVAVYYCKQSYNLWTFGQGTKLEIKVAA | |
| | PSVFIFPPSDEQLKSGTASVVCLLNNFYPREAKVQWKV | |
| | DNALQSGNSQESVTEQDSKDSTYSLSSTLTLSKADYEK | |
| | HKVYACEVTHQGLSSPVTKSFNRGEC | |
| Humanized 16B5 | QVQLVQSGAEVKKPGATVKISCKVSGFNIKDIYMHWV | 12 |
| heavy chain | QQAPGKGLEWMGRIDPAYGNTKYDPKFQGRVTITADT | |
| | STDTAYMELSSLRSEDTAVYYCArRYDTAMDYwgqgtlvt | |
| | vSSASTKGPSVFPLAPCSRSTSESTAALGCLVKDYFPEPV | |
| | TVSWNSGALTSGVHTFPAVLQSSGLYSLSSVVTVPSSN | |
| | FGTQTYTCNVDHKPSNTKVDKTVERKCCVECPPCPAPP | |
| | VAGPSVFLFPPKPKDTLMISRTPEVTCVVVDVSHEDPE | |
| | VQFNWYVDGVEVHNAKTKPREEQFNSTFRVVSVLTVV | |
| | HQDWLNGKEYKCKVSNKGLPAPIEKTISKTKGQPREPQ | |
| | VYTLPPSREEMTKNQVSLTCLVKGFYPSDIAVEWESNG | |
| | QPENNYKTTPPMLDSDGSFFLYSKLTVDKSRWQQGNV | |
| | FSCSVMHEALHNHYTQKSLSLSPGK | |
| Humanized16B5 | ATGGTGCTGCAGACCCAGGTGTTCATCTCCCTGCTGC | 13 |
| light chain | TGTGGATCTCCGGCGCCTACGGCGACATCGTGATGA | |
| nucleic acid | CCCAGTCCCCCGACTCCCTGGCCGTGTCCCTGGGCGA | |
| | GAGAGCCACCATCAACTGCAAGTCCTCCCAGTCCCT | |
| | GCTGAACTCCCGGACCCGGAAGAACTACCTGGCCTG | |
| | GTATCAGCAGAAGCCTGGCCAGCCTCCTAAGCTGCT | |
| | GATCTACTGGGCCTCCACCCGGGAGTCCGGCGTGCC | |
| | TGACCGGTTCTCCGGCTCCGGCAGCGGCACCGACTT | |
| | CACCCTGACCATCAGCTCCCTGCAGGCCGAGGACGT | |
| | GGCCGTGTACTACTGCAAGCAGTCCTACAACCTGTG | |
| | GACCTTCGGCCAGGGCACCAAGCTGGAGATCAAGCG | |
| | GACTGTGGCTGCACCATCTGTCTTCATCTTCCCGCCA | |
| | TCTGATGAGCAGTTGAAATCTGGAACTGCCTCTGTTG | |
| | TGTGCCTGCTGAATAACTTCTATCCCAGAGAGGCCA | |
| | AAGTACAGTGGAAGGTGGATAACGCCCTCCAATCGG | |
| | GTAACTCCCAGGAGAGTGTCACAGAGCAGGACAGCA | |
| | AGGACAGCACCTACAGCCTCAGCAGCACCCTGACGC | |
| | TGAGCAAAGCAGACTACGAGAAACACAAAGTCTAC | |
| | GCCTGCGAAGTCACCCATCAGGGCCTGAGCTCGCCC | |
| | GTCACAAAGAGCTTCAACAGGGGAGAGTGTTAG | |
| Humanized 16B5 | ATGGACTGGACCTGGCGGATCCTGTTCCTGGTGGCC | 14 |
| heavy chain | GCTGCTACCGGCACCCACGCCCAGGTGCAGCTGGTG | |
| nucleic acid | CAGTCTGGCGCCGAGGTGAAGAAGCCTGGCGCCACC | |
| | GTCAAGATCAGCTGCAAGGTGTCCGGCTTCAACATC | |
| | AAGGACATCTACATGCACTGGGTGCAGCAGGCTCCA | |
| | GGCAAGGGACTGGAGTGGATGGGCCGGATCGACCCT | |
| | GCCTACGGCAACACCAAGTACGACCCTAAGTTCCAG | |
| | GGCCGGGTGACCATCACCGCCGACACCTCCACCGAC | |
| | ACCGCCTACATGGAACTGTCCTCCCTGCGGTCCGAG | |
| | GACACCGCCGTGTACTACTGCGCCCGGAGATACGAC | |
| | ACCGCCATGGATTACTGGGGCCAGGGCACCCTGGTG | |
| | ACCGTGTCCTCCGCTTCCACCAAGGGCCCATCGGTCT | |
| | TCCCCCTGGCGCCCTGCTCCAGGAGCACCTCCGAGA | |
| | GCACAGCGGCCCTGGGCTGCCTGGTCAAGGACTACT | |
| | TCCCCGAACCGGTGACGGTGTCGTGGAACTCAGGCG | |
| | CTCTGACCAGCGGCGTGCACACCTTCCCAGCTGTCCT | |
| | ACAGTCCTCAGGACTCTACTCCCTCAGCAGCGTGGT | |
| | GACCGTGCCCTCCAGCAACTTCGGCACCCAGACCTA | |
| | CACCTGCAACGTAGATCACAAGCCCAGCAACACCAA | |
| | GGTGGACAAGACAGTTGAGCGCAAATGTTGTGTCGA | |
| | GTGCCCACCGTGCCCAGCACCACCTGTGGCAGGACC | |
| | GTCAGTCTTCCTCTTCCCCCCAAAACCCAAGGACACC | |
| | CTCATGATCTCCCGGACCCCTGAGGTCACGTGCGTG | |
| | GTGGTGGACGTGAGCCACGAAGACCCCGAGGTCCAG | |
| | TTCAACTGGTACGTGGACGGCGTGGAGGTGCATAAT | |
| | GCCAAGACAAAGCCACGGGAGGAGCAGTTCAACAG | |
| | CACGTTCCGTGTGGTCAGCGTCCTCACCGTTGTGCAC | |
| | CAGGACTGGCTGAACGGCAAGGAGTACAAGTGCAA | |
| | GGTCTCCAACAAAGGCCTCCCAGCCCCCATCGAGAA | |
| | AACCATCTCCAAAACCAAAGGGCAGCCCCGAGAACC | |
| | ACAGGTGTACACCCTGCCCCCATCCCGGGAGGAGAT | |
| | GACCAAGAACCAGGTCAGCCTGACCTGCCTGGTCAA | |
| | AGGCTTCTACCCCAGCGACATCGCCGTGGAGTGGGA | |
| | GAGCAATGGGCAGCCGGAGAACAACTACAAGACCA | |
| | CACCTCCCATGCTGGACTCCGACGGCTCCTTCTTCCT | |
| | CTACAGCAAGCTCACCGTGGACAAGAGCAGGTGGCA | |
| | GCAGGGGAACGTCTTCTCATGCTCCGTGATGCATGA | |
| | GGCTCTGCACAACCACTACACGCAGAAGAGCCTCTC | |
| | CCTGTCTCCGGGTAAATGA | |
| 21B12 CDRL1 | KSSQSLLYSSNQKNYLA | 15 |
| 21B12 CDRL2 | WASTRES | 16 |
| 21B12 CDRL3 | QQYYIYPRT | 17 |
| 21B12 | GYTFTNYGMH | 18 |
| CDRH1_v1 | | |
| 21B12 | WINTYTGEPTYADDFKG | 19 |
| CDRH2_v1 | | |
| 21B12 | DGFLYFFDY | 20 |
| CDRH3_v1 | | |
| 21B12 | GYTFTNYG | 38 |
| CDRH1_v2 | | |
| 21B12 | INTYTGEP | 39 |
| CDRH2_v2 | | |
| 21B12 | DGFLYFFDY | 40 |
| CDRH3_v2 | | |
| Murine 21B12 | DIVMSQSPSSLAVSVGEKVTMSCKSSQSLLYSSNQKNY | 21 |
| VL | LAWYQQRPGQSPKLLIYWASTRESGVPDRFTGSGSGTD | |
| | FTLTISSVKAEDLAVYYCQQYYIYPRTFGGGTKLEIK | |
| Murine 21B12 | QIQLVQSGPELKKPGETVKISCKASGYTFTNYGMHWV | 22 |
| VH | KQAPGKGLKWMGWINTYTGEPTYADDFKGRFAFSLET | |
| | SASTAYLQINNLKNEDTATYFCARDGFLYFFDYWGQG | |
| | TTLTVSS | |
| Humanized | DIVMTQSPDSLAVSLGERATINCKSSQSLLYSSNQKNYL | 23 |
| 21B12 VL | AWYQQKPGQPPKLLIYWASTRESGVPDRFSGSGSGTDF | |
| | TLTISSLQAEDVAVYYCQQYYIYPRTFGQGTKLEIK | |
| Humanized | QVQLVQSGSELKKPGASVKVSCKASGYTFTNYGMHW | 24 |
| 21B12 VH | VRQAPGQGLEWMGWINTYTGEPTYADDFKGRFVFSLD | |
| | TSVSTAYLQISSLKAEDTAVYYCARDGFLYFFDYWGQ | |
| | GTLVTVSS | |
| Humanized | DIVMTQSPDSLAVSLGERATINCKSSQSLLYSSNQKNYL | 25 |
| 21B12 light chain | AWYQQKPGQPPKLLIYWASTRESGVPDRFSGSGSGTDF | |
| | TLTISSLQAEDVAVYYCQQYYIYPRTFGQGTKLEIKRTV | |
| | AAPSVFIFPPSDEQLKSGTASVVCLLNNFYPREAKVQW | |
| | KVDNALQSGNSQESVTEQDSKDSTYSLSSTLTLSKADY | |
| | EKHKVYACEVTHQGLSSPVTKSFNRGEC | |
| Humanized | QVQLVQSGSELKKPGASVKVSCKASGYTFTNYGMHW | 26 |
| 21B12 heavy | VRQAPGQGLEWMGWINTYTGEPTYADDFKGRFVFSLD | |
| chain | TSVSTAYLQISSLKAEDTAVYYCARdGFLYFFDYWGQG | |
| | TLVTVSSASTKGPSVFPLAPCSRSTSESTAALGCLVKDY | |
| | FPEPVTVSWNSGALTSGVHTFPAVLQSSGLYSLSSVVT | |
| | VPSSNFGTQTYTCNVDHKPSNTKVDKTVERKCCVECPP | |
| | CPAPPVAGPSVFLFPPKPKDTLMISRTPEVTCVVVDVSH | |
| | EDPEVQFNWYVDGVEVHNAKTKPREEQFNSTFRVVSV | |
| | LTVVHQDWLNGKEYKCKVSNKGLPAPIEKTISKTKGQ | |
| | PREPQVYTLPPSREEMTKNQVSLTCLVKGFYPSDIAVE | |
| | WESNGQPENNYKTTPPMLDSDGSFFLYSKLTVDKSRW | |
| | QQGNVFSCSVMHEALHNHYTQKSLSLSPGK | |
| Humanized | ATGGTGCTGCAGACCCAGGTGTTCATCTCCCTGCTGC | 27 |
| 21B12 light chain | TGTGGATCTCTGGCGCCTACGGCGACATCGTGATGA | |
| nucleic acid | CCCAGTCCCCCGACTCTCTGGCTGTGTCCCTGGGCGA | |
| | GCGGGCCACCATCAACTGCAAGTCCTCCCAGTCCCT | |
| | GCTGTACTCCTCCAACCAGAAGAACTACCTGGCCTG | |
| | GTATCAGCAGAAGCCTGGCCAGCCTCCTAAGCTGCT | |
| | GATCTACTGGGCCTCCACCCGGGAATCTGGCGTGCC | |
| | TGACCGGTTCTCCGGCTCTGGCTCCGGCACCGACTTC | |
| | ACCCTGACCATCAGCTCCCTGCAGGCCGAGGACGTG | |
| | GCCGTGTACTACTGCCAGCAGTACTACATCTACCCTC | |
| | GGACCTTCGGCCAGGGCACCAAGCTGGAAATCAAGC | |
| | GGACCGTGGCCGCTCCTTCCGTGTTCATCTTCCCCCC | |
| | TTCCGACGAGCAGCTGAAGTCCGGCACCGCCTCTGT | |
| | GGTGTGCCTGCTGAACAACTTCTACCCCCGGGAGGC | |
| | CAAGGTGCAGTGGAAGGTGGACAACGCCCTGCAGTC | |
| | CGGCAACTCCCAGGAATCCGTCACCGAGCAGGACTC | |
| | CAAGGACTCTACCTACTCCCTGTCCTCCACCCTGACC | |
| | CTGTCCAAGGCCGACTACGAGAAGCACAAGGTGTAC | |
| | GCCTGCGAAGTGACCCACCAGGGCCTGTCCTCTCCC | |
| | GTGACCAAGTCCTTCAACCGGGGCGAGTGCTGA | |
| Humanized | ATGGACTGGACCTGGCGGATCCTGTTTCTGGTGGCC | 28 |
| 21B12 heavy | GCTGCTACCGGCACACACGCCCAGGTGCAGCTGGTG | |
| chain nucleic acid | CAGTCCGGCTCCGAGCTGAAGAAACCTGGCGCCTCC | |
| | GTGAAGGTGTCCTGCAAGGCCTCCGGCTACACCTTC | |
| | ACCAACTACGGCATGCACTGGGTGCGCCAGGCACCT | |
| | GGACAGGGACTGGAATGGATGGGCTGGATCAACACC | |
| | TACACCGGCGAGCCTACCTACGCCGACGACTTCAAG | |
| | GGCAGATTCGTGTTCTCCCTGGACACCTCCGTGTCCA | |
| | CCGCCTACCTGCAGATCTCCTCCCTGAAGGCCGAGG | |
| | ACACCGCCGTGTACTACTGCGCCAGGGACGGCTTCC | |
| | TGTACTTCTTCGACTACTGGGGCCAGGGCACCCTGGT | |
| | GACCGTGTCCTCTGCCTCCACCAAGGGCCCTTCCGTG | |
| | TTCCCTCTGGCCCCTTGCTCCCGGTCCACCTCTGAGT | |
| | CTACCGCCGCTCTGGGCTGCCTGGTGAAGGACTACTT | |
| | CCCTGAGCCTGTGACAGTGTCCTGGAACTCTGGCGC | |
| | CCTGACCTCTGGCGTGCACACCTTCCCTGCCGTGCTG | |
| | CAGTCCTCCGGCCTGTACTCCCTGTCCTCCGTGGTGA | |
| | CAGTGCCTTCCTCCAACTTCGGCACCCAGACCTACAC | |
| | CTGCAACGTGGACCACAAGCCTTCCAACACCAAGGT | |
| | GGACAAGACCGTGGAGCGGAAGTGCTGCGTGGAGT | |
| | GCCCTCCTTGTCCTGCTCCTCCTGTGGCTGGCCCTAG | |
| | CGTGTTCCTGTTCCCTCCTAAGCCTAAGGACACCCTG | |
| | ATGATCTCCCGGACCCCTGAAGTGACCTGCGTGGTG | |
| | GTGGACGTGTCCCACGAGGACCCTGAGGTGCAGTTC | |
| | AATTGGTACGTGGACGGCGTGGAGGTGCACAACGCC | |
| | AAGACCAAGCCTCGGGAGGAACAGTTCAACTCCACC | |
| | TTCCGGGTGGTGTCCGTGCTGACCGTGGTGCACCAG | |
| | GACTGGCTGAACGGCAAAGAATACAAGTGCAAGGT | |
| | GTCCAACAAGGGCCTGCCTGCCCCTATCGAAAAGAC | |
| | CATCTCTAAGACCAAGGGCCAGCCTCGCGAGCCTCA | |
| | GGTGTACACCCTGCCTCCCTCCCGCGAGGAAATGAC | |
| | CAAGAACCAGGTGTCCCTGACCTGTCTGGTGAAGGG | |
| | CTTCTACCCTTCCGATATCGCCGTGGAGTGGGAGTCT | |
| | AACGGCCAGCCTGAGAACAACTACAAGACCACCCCT | |
| | CCTATGCTGGACTCCGACGGCTCCTTCTTCCTGTACA | |
| | GCAAGCTGACAGTGGACAAGTCCCGGTGGCAGCAGG | |
| | GCAACGTGTTCTCCTGCTCCGTGATGCACGAGGCCCT | |
| | GCACAACCACTACACCCAGAAGTCCCTGTCCCTGTCT | |
| | CCTGGCAAGTGA | |
| 20E11 variable | DIVLTLSPASLAVSLGQRATISCRASQSVNSSNYSYMH | 29 |
| light chain | WYQQKPGQPPKLLIKYASNLESGVPARFSGSGSGTHFT | |
| | LNIHPVEEEDTATYYCQHSWEIPWTFGGGTKLEIK | |
| 20E11 variable | QIQLVQSGPELKKPGETVKISCKASGYTFTDYSMHWVK | 30 |
| heavy chain | QAPGKGLKWMGWINTETGEPTYADDFKGRFAFSLETS | |
| | ASTAYLQINNLKNEDTATYFCARTGSSGYFDCWGQGT | |
| | TLTVSS | |
| 11E2 variable | ENVLTQSPAIMSASPGEKVTMTCSASSSVSYMHWYQQ | 31 |
| light chain Gr1 | KSSTSPKLWIYDTSKLASGVPGRFSGSGSGNSYSLTISS | |
| | MEAEDVATYYCFQGSGYPFTFGSGTKLEIK | |
| 11E2 variable | DIQMTQSPSSLSASLGGKVTITCKASQDINKYIAWYQH | 32 |
| light chain GR2 | KPGKGPRLLIHYTSTLQPGIPSRFSGSGSGRDYSFSISNL | |
| | EPEDIATYYCLQYDNLLRTFGGGTKLEIK | |
| 11E2 variable | EVQLQQSGPELEKPGASVKISCKASGYSFTGYNMNWV | 33 |
| heavy chain | KONNGKSLEWIGNIDPYYGTPNYNQKFKGKATLTVDK | |
| | SSSTAYMQLKSLTSEDSAVYYCALNSLLRLNAMDYWG | |
| | QGTSVTVSS | |
| 16C11 variable | EVQLQQSGPELGKPGASVKISCKASGYSFTGYNMYWV | 34 |
| heavy chain | KQSHRKSLEWIGYIDPYNGDTSYNQKSKGKATLTADR | |
| | SSSTAYMHLNSLTSEDSGIYYCARGAYGSSYAYWGQG | |
| | TLVAVSA | |
| Amino acids 421 | LTQGEDQYYLRVTTVASHTSDSDVPSGVTEVVVKLFD | 41 |
| and 443 of a C- | SDPITVTVPVEVSRKNPKFMETV | |
| terminal portion | AEKALQEYRKKHREE | |
| of a ß-subunit of | | |
| human clusterin | | |

1. A method of treating a subject having cancer, the method comprising administering an anti-cancer therapy that comprises an anti-clusterin antibody or antigen binding fragment thereof to the subject, isolating and expanding tumor infiltrating lymphocytes (TILs) from the subject's tumor and reinfusing a preparation of TILs to the subject.

2. A method of treating a subject having cancer, the method comprising administering a preparation of tumor infiltrating lymphocytes (TILs) isolated from the subject's tumor, wherein the subject has received prior treatment with an anti-cancer therapy that comprises an anti-clusterin antibody or antigen binding fragment thereof.

3. The method of claim 1 or 2, wherein preparation of TILs is isolated and expanded by an in vitro or ex vivo method of generating tumor infiltrating lymphocytes.

4. The method of any one of claims 1 to 3, wherein the anti-cancer therapy is an anti-clusterin antibody or an antigen binding fragment thereof as a single agent.

5. The method of claims 1 to 3, wherein the anti-cancer therapy is a combination therapy comprising an anti-clusterin antibody or an antigen binding fragment thereof and a chemotherapeutic agent.

6. The method of claim 4, wherein the chemotherapeutic agent is selected from an alkylating agent, an anti-metabolite, an alkaloid, an anti-tumor antibiotic or combination thereof.

7. The method of claim 5, wherein the chemotherapeutic agent is docetaxel or paclitaxel.

8. The method of any one of the preceding claims, wherein the tumor is resectable.

9. The method of any one of the preceding claims, wherein the subject has a functional immune system.

10. The method of any one of the preceding claims, wherein the preparation of TILs is obtained from a tumor or tumor fragments isolated by biopsy.

11. The method of claim 3, wherein the in vitro or ex vivo method of generating the preparation of tumor infiltrating lymphocytes comprises a step of contacting tumor fragments with an anti-clusterin antibody or antigen binding fragment thereof.

12. The method of claim 11, wherein the anti-clusterin antibody or an antigen binding fragment thereof is present and/or maintained during one or more phases of the method of generating the preparation of tumor infiltrating lymphocytes.

13. The method of any one of the preceding claims, wherein the preparation of TILs are not genetically modified.

14. The method of any one of the preceding claims, wherein the preparation of TILs comprises TILs that are genetically modified.

15. The method of claim 14, wherein the preparation of TILs comprises TILs that express a chimeric antigen receptor.

16. The method of claim 14, wherein the preparation of TILs comprises TILs that express a transgenic T-cell receptor.

17. The method of any one of the preceding claims, wherein the subject's tumor is a primary tumor.

18. The method of any one of the preceding claims, wherein the subject's tumor is a metastasis.

19. The method of any one of the preceding claims, wherein the anti-clusterin antibody or antigen binding fragment thereof is administered at a dose and/or an administration interval and/or for a treatment period sufficient to result in infiltration of immune cells in the tumor microenvironment.

20. The method any one of claims 7 to 19, wherein docetaxel is administered at a dose and/or an administration interval and/or for a treatment period sufficient to allow chemotherapy-induced immunogenic modulation of tumor.

21. The method of any of the preceding claims, wherein the anti-clusterin antibody or antigen binding fragment thereof comprises a light chain variable region comprising the complementarity determining regions (CDRs) of the light chain variable region set forth in SEQ ID NO:9 and a heavy chain variable region comprising the CDRs of the heavy chain variable region set forth in SEQ ID NO:10.

22. The method of any of the preceding claims, wherein the anti-clusterin antibody or antigen binding fragment thereof comprises a light chain variable region having an amino acid sequence having at least 80% identity with the amino acid sequence set forth in SEQ ID NO:9 and a heavy chain variable region having an amino acid sequence at least 80% identity with the amino acid sequence set forth in SEQ ID NO:10.

23. The method of any of the preceding claims, wherein the anti-clusterin antibody or antigen binding fragment thereof comprises a light chain having an amino acid sequence having at least 80% identity with the amino acid sequence set forth in SEQ ID NO:11 and a heavy chain having an amino acid sequence having at least 80% identity with the amino acid sequence set forth in SEQ ID NO: 12.

24. The method of any of the preceding claims, wherein the antibody or antigen binding fragment thereof is capable of competing with an antibody comprising a light chain variable region having an amino acid sequence set forth in SEQ ID NO:9 and a heavy chain variable region having an amino acid sequence set forth in SEQ ID NO:10 for the binding of clusterin.

25. The method of any of the preceding claims, wherein the preparation of TILs comprises CD4⁺ T cells.

26. The method of any of the preceding claims, wherein the preparation of TILs comprises CD8⁺ T cells.

27. The method of claim 26, wherein the preparation of TILs comprises at least 50% of CD8+ lymphocytes.

28. The method of any one of the preceding claims, wherein the preparation of TILs comprises B cells.

29. The method of any one of the preceding claims, wherein the preparation of TILs comprises NK cells.

30. The method of any one of the preceding claims, wherein the preparation of TILs comprises NK T cells.

31. The method of any one of the preceding claims, wherein the preparation of TILs is selected for tumor antigen recognition.

32. The method of any one of the preceding claims, wherein the preparation of TILs secretes intermediate to high levels of INFγ.

33. The method of any one of the preceding claims, wherein the anti-clusterin antibody or antigen binding fragment thereof is administered at a dose of between approximately 3 mg/kg to approximately 20 mg/kg prior to isolation of TILs or after infusion of TILs.

34. The method of any one of the preceding claims, wherein the anti-clusterin antibody or antigen binding fragment thereof is administered at a dose of approximately 6 mg/kg.

35. The method of any one of the preceding claims, wherein the anti-clusterin antibody or antigen binding fragment thereof is administered at a dose of approximately 9 mg/kg.

36. The method of any one of the preceding claims, wherein the anti-clusterin antibody or antigen binding fragment thereof is administered at a dose of approximately 12 mg/kg.

37. The method of any one of claims 7 to 36, wherein docetaxel is administered at a dose of between approximately 60 mg/m² to approximately 100 mg/m² prior to isolation of TILs or after infusion of TILs.

38. The method of any one of claims 7 to 37, wherein docetaxel is administered at a dose of approximately 60 mg/m².

39. The method of any one of claims 7 to 37, wherein docetaxel is administered at a dose of approximately 75 mg/m².

40. The method of any one of claims 7 to 37, wherein the subject is treated with the anti-clusterin antibody or antigen binding fragment thereof at a dose of approximately 12 mg/kg once weekly and docetaxel at a dose of approximately 75 mg/m² once every three weeks.

41. The method of any one of claims 7 to 37, wherein the subject is treated with the anti-clusterin antibody or antigen binding fragment thereof at a dose of approximately 12 mg/kg once weekly and docetaxel at a dose of approximately 60 mg/m² once every three weeks.

42. The method of any one of claims 7 to 37, wherein the subject is treated with the anti-clusterin antibody or antigen binding fragment thereof at a dose of approximately 9 mg/kg once weekly and docetaxel at a dose of approximately 75 mg/m² once every three weeks.

43. The method of any one of claims 7 to 37, wherein the subject is treated with the anti-clusterin antibody or antigen binding fragment thereof at a dose of approximately 9 mg/kg once weekly and docetaxel at a dose of approximately 60 mg/m² once every three weeks.

44. The method of any one of claims 7 to 37, wherein the subject is treated with the anti-clusterin antibody or antigen binding fragment thereof at a dose of approximately 6 mg/kg once weekly and docetaxel at a dose of approximately 75 mg/m² once every three weeks.

45. The method of any one of claims 7 to 37, wherein the subject is treated with the anti-clusterin antibody or antigen binding fragment thereof at a dose of approximately 6 mg/kg once weekly and docetaxel at a dose of approximately 60 mg/m² once every three weeks.

46. The method of any one of claims 7 to 37, wherein the subject is treated with the anti-clusterin antibody or antigen binding fragment thereof at a dose of approximately 3 mg/kg once weekly and docetaxel at a dose of approximately 75 mg/m² once every three weeks.

47. The method of any one of claims 7 to 37, wherein the subject is treated with the anti-clusterin antibody or antigen binding fragment thereof at a dose of approximately 3 mg/kg once weekly and docetaxel at a dose of approximately 60 mg/m² once every three weeks.

48. The method of any one of claims 7 to 47, wherein the anti-clusterin antibody or antigen binding fragment thereof and docetaxel are administered on same day.

49. The method of any one of claims 7 to 48, wherein the anti-clusterin antibody or antigen binding fragment thereof and/or docetaxel is administered by infusion over approximately a 1-hour time frame.

50. The method of any one of claims 7 to 49, wherein the anti-clusterin antibody or antigen binding fragment thereof and docetaxel are administered for at least two cycles of treatment prior to isolation of TILs.

51. The method of any one of claims 7 to 49, wherein the anti-clusterin antibody or antigen binding fragment thereof and docetaxel are administered for at least two cycles of treatment prior after infusion of TILs.

52. The method of any one of the preceding claims, wherein the subject has a carcinoma.

53. The method of any one of the preceding claims, wherein the carcinoma is metastatic.

54. The method of any one of the preceding claims, wherein the subject has an endometrial cancer, a breast cancer, a liver cancer, a prostate cancer, a renal cancer, a bladder cancer, a cervical cancer, an ovarian cancer, a colorectal cancer, a pancreatic cancer, a lung cancer, a gastric cancer, a head and neck cancer, a thyroid cancer, a cholangiocarcinoma, a mesothelioma or a melanoma.

55. The method of any one of the preceding claims, wherein the subject has a metastatic endometrial cancer, a metastatic breast cancer, a metastatic liver cancer, a metastatic prostate cancer, a metastatic renal cancer, a metastatic bladder cancer, a metastatic cervical cancer, a metastatic ovarian cancer, a metastatic colorectal cancer, a metastatic pancreatic cancer, a metastatic lung cancer, a metastatic gastric cancer, a metastatic head and neck cancer, a metastatic thyroid cancer, a metastatic cholangiocarcinoma, a metastatic mesothelioma or a metastatic melanoma.

56. The method of any one of the preceding claims, wherein the subject is not immunosuppressed or has not received an immunosuppressive medication within 7 days prior to treatment with the anti-clusterin antibody or antigen binding fragment thereof or prior to treatment with the anti-clusterin antibody or antigen binding fragment thereof and docetaxel combination therapy.

57. The method of any one of the preceding claims, wherein the subject receives lymphocyte-depleting preparative regimen prior to infusion of TILs.

58. The method of any one of the preceding claims, wherein the subject is a human subject.

59. A preparation of tumor infiltrating lymphocytes (TILs) obtained by the method of any one of claims 1 to 58.

60. A preparation of tumor infiltrating lymphocytes (TILs) obtained by a method of treating a subject having cancer with an anti-cancer therapy that comprises an anti-clusterin antibody or antigen binding fragment thereof, isolating and expanding tumor infiltrating lymphocytes (TILs) from the subject's tumor.

61. The preparation of TILs of claim 60, wherein the subject has been treated or is treated with an anti-clusterin antibody or an antigen binding fragment thereof as a single agent or in combination therapy with a chemotherapeutic agent.

62. The preparation of TILs of claim 60 or 61, wherein the preparation of TILs is not genetically modified.

63. The preparation of TILs of claim 60 or 61, wherein the preparation of TILs comprises TILs that are genetically modified.

64. The preparation of TILs of claim 63, wherein the preparation of TILs comprises TILs that express a chimeric antigen receptor.

65. The preparation of TILs of claim 63, wherein the preparation of TILs comprises TILs that express a transgenic T-cell receptor.

66. The preparation of TILs of any one of claims 60 to 65, wherein the preparation of TILs is provided in an infusion bag.

67. The preparation of TILs of any one of claims 60 to 66, wherein the preparation of TILs comprises a majority of CD45⁺ cells.

68. The preparation of TILs of any one of claims 60 to 67, wherein the preparation of TILs comprises a majority of CD3⁺ cells.

69. The preparation of TILs of any one of claims 60 to 68, wherein the preparation of TILs comprises a majority of CD4⁺ cells.

70. The preparation of TILs of any one of claims 60 to 68, wherein the preparation of TILs comprises a majority of CD8⁺ cells.

71. The preparation of TILs of claim 69, wherein the preparation of TILs comprises at least 50% of CD8+ lymphocytes.

72. The preparation of TILs of any one of claims 60 to 66, wherein the preparation of TILs comprises TILs that secrete intermediate to high levels of INFγ.

73. The preparation of TILs of any one of claims 60 to 66, wherein the preparation of TILs comprises a majority of cells that are CD4⁺ or CD8⁺ cells.

74. The preparation of TILs of any one of claims 60 to 72, wherein the preparation of TILs is for use in adoptive cell therapy.

75. An article of manufacture comprising the preparation of TILs of any one of the preceding claims.

76. A tumor infiltrating lymphocytes (TILs) culture obtained by the method of any one of claims 1 to 58.


SEQUENCE LISTING (41 sequences)

SEQ ID NO: 1 - protein, 17 aa, Artificial Sequence
  16B5 CDRL1
KSSQSLLNSRTRKNYLA

SEQ ID NO: 2 - protein, 7 aa, Artificial Sequence
  16B5 CDRL2
WASTRES

SEQ ID NO: 3 - protein, 8 aa, Artificial Sequence
  16B5 CDRL3
KQSYNLWT

SEQ ID NO: 4 - protein, 10 aa, Artificial Sequence
  16B5 CDRH1-V1
GFNIKDIYMH

SEQ ID NO: 5 - protein, 17 aa, Artificial Sequence
  16B5 CDRH2-V1
RIDPAYGNTKYDPKFQG

SEQ ID NO: 6 - protein, 8 aa, Artificial Sequence
  16B5 CDRH3-V1
RYDTAMDY

SEQ ID NO: 7 - protein, 112 aa, Artificial Sequence
  Murine 16B5 VL
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLWTFGGGTKLEFK

SEQ ID NO: 8 - protein, 117 aa, Artificial Sequence
  Murine 16B5 VH
EVQLQQSGAELVKPGASVRLSCTTSGFNIKDIYMHWVKQRPEQGLEWIGRIDPAYGNTKYDPKFQGKATITADTSSNTAYLQLSSLTSEDTAVYYCARRYDTAMDYWGQGTSVTVSS

SEQ ID NO: 9 - protein, 112 aa, Artificial Sequence
  Humanized 16B5 VL
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYNLWTFGQGTKLEIK

SEQ ID NO: 10 - protein, 117 aa, Artificial Sequence
  Humanized 16B5 VH
QVQLVQSGAEVKKPGATVKISCKVSGFNIKDIYMHWVQQAPGKGLEWMGRIDPAYGNTKYDPKFQGRVTITADTSTDTAYMELSSLRSEDTAVYYCARRYDTAMDYWGQGTLVTVSS

SEQ ID NO: 11 - protein, 217 aa, Artificial Sequence
  Humanized 16B5 light chain
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYNLWTFGQGTKLEIKVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 443 aa, Artificial Sequence
  Humanized 16B5 heavy chain
QVQLVQSGAEVKKPGATVKISCKVSGFNIKDIYMHWVQQAPGKGLEWMGRIDPAYGNTKYDPKFQGRVTITADTSTDTAYMELSSLRSEDTAVYYCARRYDTAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - DNA, 720 bp, Artificial Sequence
  Humanized 16B5 light chain nucleic acid
atggtgctgcagacccaggtgttcatctccctgctgctgtggatctccggcgcctacggcgacatcgtgatgacccagtcccccgactccctggccgtgtccctgggcgagagagccaccatcaactgcaagtcctcccagtccctgctgaactcccggacccggaagaactacctggcctggtatcagcagaagcctggccagcctcctaagctgctgatctactgggcctccacccgggagtccggcgtgcctgaccggttctccggctccggcagcggcaccgacttcaccctgaccatcagctccctgcaggccgaggacgtggccgtgtactactgcaagcagtcctacaacctgtggaccttcggccagggcaccaagctggagatcaagcggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 14 - DNA, 1389 bp, Artificial Sequence
  Humanized 16B5 heavy chain nucleic acid
atggactggacctggcggatcctgttcctggtggccgctgctaccggcacccacgcccaggtgcagctggtgcagtctggcgccgaggtgaagaagcctggcgccaccgtcaagatcagctgcaaggtgtccggcttcaacatcaaggacatctacatgcactgggtgcagcaggctccaggcaagggactggagtggatgggccggatcgaccctgcctacggcaacaccaagtacgaccctaagttccagggccgggtgaccatcaccgccgacacctccaccgacaccgcctacatggaactgtcctccctgcggtccgaggacaccgccgtgtactactgcgcccggagatacgacaccgccatggattactggggccagggcaccctggtgaccgtgtcctccgcttccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 15 - protein, 17 aa, Artificial Sequence
  21B12 CDRL1
KSSQSLLYSSNQKNYLA

SEQ ID NO: 16 - protein, 7 aa, Artificial Sequence
  21B12 CDRL2
WASTRES

SEQ ID NO: 17 - protein, 9 aa, Artificial Sequence
  21B12 CDRL3
QQYYIYPRT

SEQ ID NO: 18 - protein, 10 aa, Artificial Sequence
  21B12 CDRH1-v1
GYTFTNYGMH

SEQ ID NO: 19 - protein, 17 aa, Artificial Sequence
  21B12 CDRH2-v1
WINTYTGEPTYADDFKG

SEQ ID NO: 20 - protein, 9 aa, Artificial Sequence
  21B12 CDRH3-v1
DGFLYFFDY

SEQ ID NO: 21 - protein, 113 aa, Artificial Sequence
  Murine 21B12 VL
DIVMSQSPSSLAVSVGEKVTMSCKSSQSLLYSSNQKNYLAWYQQRPGQSPKLLIYWASTRESGVPDRFTGSGSGTDFTLTISSVKAEDLAVYYCQQYYIYPRTFGGGTKLEIK

SEQ ID NO: 22 - protein, 118 aa, Artificial Sequence
  Murine 21B12 VH
QIQLVQSGPELKKPGETVKISCKASGYTFTNYGMHWVKQAPGKGLKWMGWINTYTGEPTYADDFKGRFAFSLETSASTAYLQINNLKNEDTATYFCARDGFLYFFDYWGQGTTLTVSS

SEQ ID NO: 23 - protein, 113 aa, Artificial Sequence
  Humanized 21B12 VL
DIVMTQSPDSLAVSLGERATINCKSSQSLLYSSNQKNYLAWYQQKPGQPPKLLIYWASTRESGVPDRFSGSGSGTDFTLTISSLQAEDVAVYYCQQYYIYPRTFGQGTKLEIK

SEQ ID NO: 24 - protein, 118 aa, Artificial Sequence
  Humanized 21B12 VH
QVQLVQSGSELKKPGASVKVSCKASGYTFTNYGMHWVRQAPGQGLEWMGWINTYTGEPTYADDFKGRFVFSLDTSVSTAYLQISSLKAEDTAVYYCARDGFLYFFDYWGQGTLVTVSS

SEQ ID NO: 25 - protein, 220 aa, Artificial Sequence
  Humanized 21B12 light chain
DIVMTQSPDSLAVSLGERATINCKSSQSLLYSSNQKNYLAWYQQKPGQPPKLLIYWASTRESGVPDRFSGSGSGTDFTLTISSLQAEDVAVYYCQQYYIYPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 444 aa, Artificial Sequence
  Humanized 21B12 heavy chain
QVQLVQSGSELKKPGASVKVSCKASGYTFTNYGMHWVRQAPGQGLEWMGWINTYTGEPTYADDFKGRFVFSLDTSVSTAYLQISSLKAEDTAVYYCARDGFLYFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 27 - DNA, 723 bp, Artificial Sequence
  Humanized 21B12 light chain nucleic acid
atggtgctgcagacccaggtgttcatctccctgctgctgtggatctctggcgcctacggcgacatcgtgatgacccagtcccccgactctctggctgtgtccctgggcgagcgggccaccatcaactgcaagtcctcccagtccctgctgtactcctccaaccagaagaactacctggcctggtatcagcagaagcctggccagcctcctaagctgctgatctactgggcctccacccgggaatctggcgtgcctgaccggttctccggctctggctccggcaccgacttcaccctgaccatcagctccctgcaggccgaggacgtggccgtgtactactgccagcagtactacatctaccctcggaccttcggccagggcaccaagctggaaatcaagcggaccgtggccgctccttccgtgttcatcttccccccttccgacgagcagctgaagtccggcaccgcctctgtggtgtgcctgctgaacaacttctacccccgggaggccaaggtgcagtggaaggtggacaacgccctgcagtccggcaactcccaggaatccgtcaccgagcaggactccaaggactctacctactccctgtcctccaccctgaccctgtccaaggccgactacgagaagcacaaggtgtacgcctgcgaagtgacccaccagggcctgtcctctcccgtgaccaagtccttcaaccggggcgagtgctga

SEQ ID NO: 28 - DNA, 1392 bp, Artificial Sequence
  Humanized 21B12 heavy chain nucleic acid
atggactggacctggcggatcctgtttctggtggccgctgctaccggcacacacgcccaggtgcagctggtgcagtccggctccgagctgaagaaacctggcgcctccgtgaaggtgtcctgcaaggcctccggctacaccttcaccaactacggcatgcactgggtgcgccaggcacctggacagggactggaatggatgggctggatcaacacctacaccggcgagcctacctacgccgacgacttcaagggcagattcgtgttctccctggacacctccgtgtccaccgcctacctgcagatctcctccctgaaggccgaggacaccgccgtgtactactgcgccagggacggcttcctgtacttcttcgactactggggccagggcaccctggtgaccgtgtcctctgcctccaccaagggcccttccgtgttccctctggccccttgctcccggtccacctctgagtctaccgccgctctgggctgcctggtgaaggactacttccctgagcctgtgacagtgtcctggaactctggcgccctgacctctggcgtgcacaccttccctgccgtgctgcagtcctccggcctgtactccctgtcctccgtggtgacagtgccttcctccaacttcggcacccagacctacacctgcaacgtggaccacaagccttccaacaccaaggtggacaagaccgtggagcggaagtgctgcgtggagtgccctccttgtcctgctcctcctgtggctggccctagcgtgttcctgttccctcctaagcctaaggacaccctgatgatctcccggacccctgaagtgacctgcgtggtggtggacgtgtcccacgaggaccctgaggtgcagttcaattggtacgtggacggcgtggaggtgcacaacgccaagaccaagcctcgggaggaacagttcaactccaccttccgggtggtgtccgtgctgaccgtggtgcaccaggactggctgaacggcaaagaatacaagtgcaaggtgtccaacaagggcctgcctgcccctatcgaaaagaccatctctaagaccaagggccagcctcgcgagcctcaggtgtacaccctgcctccctcccgcgaggaaatgaccaagaaccaggtgtccctgacctgtctggtgaagggcttctacccttccgatatcgccgtggagtgggagtctaacggccagcctgagaacaactacaagaccacccctcctatgctggactccgacggctccttcttcctgtacagcaagctgacagtggacaagtcccggtggcagcagggcaacgtgttctcctgctccgtgatgcacgaggccctgcacaaccactacacccagaagtccctgtccctgtctcctggcaagtga

SEQ ID NO: 29 - protein, 111 aa, Artificial Sequence
  20E11 variable light chain
DIVLTLSPASLAVSLGQRATISCRASQSVNSSNYSYMHWYQQKPGQPPKLLIKYASNLESGVPARFSGSGSGTHFTLNIHPVEEEDTATYYCQHSWEIPWTFGGGTKLEIK

SEQ ID NO: 30 - protein, 118 aa, Artificial Sequence
  20E11 variable heavy chain
QIQLVQSGPELKKPGETVKISCKASGYTFTDYSMHWVKQAPGKGLKWMGWINTETGEPTYADDFKGRFAFSLETSASTAYLQINNLKNEDTATYFCARTGSSGYFDCWGQGTTLTVSS

SEQ ID NO: 31 - protein, 106 aa, Artificial Sequence
  11E2 variable light chain Gr1
ENVLTQSPAIMSASPGEKVTMTCSASSSVSYMHWYQQKSSTSPKLWIYDTSKLASGVPGRFSGSGSGNSYSLTISSMEAEDVATYYCFQGSGYPFTFGSGTKLEIK

SEQ ID NO: 32 - protein, 107 aa, Artificial Sequence
  11E2 variable light chain Gr2
DIQMTQSPSSLSASLGGKVTITCKASQDINKYIAWYQHKPGKGPRLLIHYTSTLQPGIPSRFSGSGSGRDYSFSISNLEPEDIATYYCLQYDNLLRTFGGGTKLEIK

SEQ ID NO: 33 - protein, 120 aa, Artificial Sequence
  11E2 variable heavy chain
EVQLQQSGPELEKPGASVKISCKASGYSFTGYNMNWVKQNNGKSLEWIGNIDPYYGTPNYNQKFKGKATLTVDKSSSTAYMQLKSLTSEDSAVYYCALNSLLRLNAMDYWGQGTSVTVSS

SEQ ID NO: 34 - protein, 118 aa, Artificial Sequence
  16C11 variable heavy chain
EVQLQQSGPELGKPGASVKISCKASGYSFTGYNMYWVKQSHRKSLEWIGYIDPYNGDTSYNQKSKGKATLTADRSSSTAYMHLNSLTSEDSGIYYCARGAYGSSYAYWGQGTLVAVSA

SEQ ID NO: 35 - protein, 8 aa, Artificial Sequence
  16B5 CDRH1-v2
GFNIKDIY

SEQ ID NO: 36 - protein, 8 aa, Artificial Sequence
  16B5 CDRH2-v2
IDPAYGNT

SEQ ID NO: 37 - protein, 10 aa, Artificial Sequence
  16B5 CDRH3-v2
ARRYDTAMDY

SEQ ID NO: 38 - protein, 8 aa, Artificial Sequence
  21B12 CDRH1-v2
GYTFTNYG

SEQ ID NO: 39 - protein, 8 aa, Artificial Sequence
  21B12 CDRH2-v2
INTYTGEP

SEQ ID NO: 40 - protein, 9 aa, Artificial Sequence
  21B12 CDRH3-v2
DGFLYFFDY

SEQ ID NO: 41 - protein, 75 aa, Artificial Sequence
  amino acids 421-443 of C-terminal portion of beta subunit of human clusterin
LTQGEDQYYLRVTTVASHTSDSDVPSGVTEVVVKLFDSDPITVTVPVEVSRKNPKFMETVAEKALQEYRKKHREE